(19) United States
(12) Patent Application Publication
(10) Pub. No.: US 2004/0048866 A1
(43) Pub. Date: Mar. 11, 2004

(54) Indazole derivatives that are activators of soluble guanylate cyclase

(76) Inventors: Teodozyj Kolasa, Lake Villa, IL (US); Jorge D. Brioni, Vernon Hills, IL (US); Vijaya Gracias, Lindenhurst, IL (US); Masaki Nakane, Lake Bluff, IL (US); Meena V. Patel, Green Oaks, IL (US); Jeffrey Rohde, Evanston, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/375,842

(22) Filed: Feb. 26, 2003

Related U.S. Application Data
(60) Provisional application No. 60/362,724, filed on Mar. 8, 2002.

(51) Int. Cl. A61K 31/519; A61K 31/502; A61K 31/4745; A61K 31/4162; C07D 471/02; C07D 487/02

(52) U.S. Cl. 514/248; 514/262.1; 514/303; 544/236; 544/262; 546/118; 514/406; 548/361.1

(57) ABSTRACT

Compounds of formula (I)

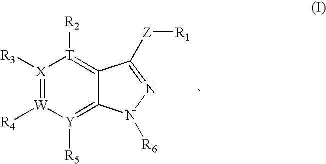

are novel indazoles useful for increasing cGMP levels in a mammal.

This application claims priority from U.S. Patent Application Ser. No. 60/362,724, filed Mar. 8, 2002, incorporated herein by reference.

### TECHNICAL FIELD

The present invention relates to novel indazoles which are activators of soluble guanylate cyclase, their preparation and their use.

### BACKGROUND OF THE INVENTION

Soluble guanylate cyclase (sGC) catalyzes the conversion of guanosine 5′-triphosphate (GTP) to cyclic guanosine 3′,5′-monophosphate (cGMP). sGC is activated by nitric oxide (NO) binding to the enzyme. In response to stimulation by NO, soluble guanylate cyclase produces cGMP which serves as a second messenger for a variety of extracellular signals from neurotransmitters and hormones by way of its interaction with intracellular targets including kinases and ion channels. cGMP causes smooth muscle relaxation by interacting with protein kinase G (PKG).

Pharmacological stimulation of sGC resulting in increased cGMP levels opens a new approach for treatment or prevention of disorders, such as cardiovascular disease, atherosclerosis, angina pectoris, diastolic dysfunction, benign prostatic hyperplasia (BPH), incontinence, sexual dysfunction, endothelial dysfunction, trombosis, diabetes, liver cirhosis, CNS disorders as cognitive disorders, Alzheimer's disease, anxiety, stress, depression, sleep disorders, migraine, cerebral ischemia, brain trauma, pain, memory and learning capabilities, associated with low levels of cGMP.

The present invention discloses novel indazole derivatives that increase cGMP levels.

### SUMMARY OF THE INVENTION

The present invention discloses indazole compounds, a method for increasing cGMP levels in a mammal using these compounds, and pharmaceutical compositions containing compounds of the present invention. More particularly, the present invention is directed to compounds of formula (I);

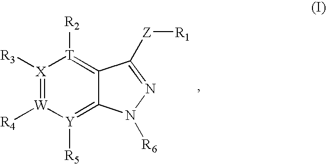

or a pharmaceutically acceptable salt, ester, amide or prodrug thereof, wherein

T, X, W, and Y are independently selected from C or N;

provided that at most, only two of T, X, W, and Y can be nitrogen at the same time;

Z is selected from O, S or N(R₇);

R₁ is selected from aryl, arylalkenyl, arylalkyl, heterocycle, heterocyclealkenyl, or heterocyclealkyl;

R₂ and R₄ are independently absent or selected from hydrogen, alkenyl, alkoxy, alkoxycarbonyl, alkyl, alkylcarbonyl, alkylcarbonyloxy, alkylthio, alkynyl, carboxy, cyano, formyl, halogen, haloalkoxy, haloalkyl, hydroxy, hydroxyalkyl, mercapto, nitro, —NZ₁Z₂, (NZ₁Z₂)carbonyl, or (NZ₁Z₂)sulfonyl, wherein Z₁ and Z₂ are independently selected from hydrogen, alkyl, alkylcarbonyl, or formyl;

R₃ and R₅ are independently absent or selected from hydrogen, alkenyl, alkoxy, alkoxycarbonyl, alkyl, alkylcarbonyl, alkylcarbonyloxy, alkylthio, alkynyl, carboxy, cyano, formyl, halogen, haloalkoxy, haloalkyl, hydroxy, hydroxyalkyl, mercapto, nitro, —NZ₁Z₂, (NZ₁Z₂)carbonyl, or (NZ₁Z₂)sulfonyl, wherein Z₁ and Z₂ are independently selected from hydrogen, alkyl, alkylcarbonyl, or formyl;

R₆ is selected from aryl, arylalkenyl, arylalkyl, heterocycle, heterocyclealkenyl, or heterocyclealkyl; and

R₇ is selected from hydrogen or alkyl.

### DETAILED DESCRIPTION OF THE INVENTION

All patents, patent applications, and literature references cited in the specification are herein incorporated by reference in their entirety. In the case of inconsistencies, the present disclosure, including definitions, will prevail.

In its principle embodiment, the present invention relates to compounds of formula (I)

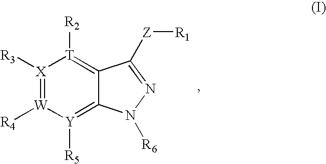

or a pharmaceutically acceptable salt, ester, amide or prodrug thereof, wherein

T, X, W, and Y are independently selected from C or N;

provided that at most, only two of T, X, W, and Y can be nitrogen at the same time;

Z is selected from O, S or N(R₇);

R₁ is selected from aryl, arylalkenyl, arylalkyl, heterocycle, heterocyclealkenyl, or heterocyclealkyl;

R₂ and R₄ are independently absent or selected from hydrogen, alkenyl, alkoxy, alkoxycarbonyl, alkyl, alkylcarbonyl, alkylcarbonyloxy, alkylthio, alkynyl, carboxy, cyano, formyl, halogen, haloalkoxy, haloalkyl, hydroxy, hydroxyalkyl, mercapto, nitro, —NZ₁Z₂, (NZ₁Z₂)carbonyl, or (NZ₁Z₂)sulfonyl, wherein Z₁ and Z₂ are independently selected from hydrogen, alkyl, alkylcarbonyl, or formyl;

R₃ and R₅ are independently absent or selected from hydrogen, alkenyl, alkoxy, alkoxycarbonyl, alkyl, alkylcarbonyl, alkylcarbonyloxy, alkylthio, alkynyl, carboxy, cyano, formyl, halogen, haloalkoxy, haloalkyl, hydroxy, hydroxyalkyl, mercapto, nitro, —NZ₁Z₂, (NZ₁Z₂)carbonyl, or (NZ₁Z₂)sulfonyl, wherein Z₁ and Z₂ are independently selected from hydrogen, alkyl, alkylcarbonyl, or formyl;

R₆ is selected from aryl, arylalkenyl, arylalkyl, heterocycle, heterocyclealkenyl, or heterocyclealkyl; and

R₇ is selected from hydrogen or alkyl.

In another embodiment, compounds of the present invention have formula (I) wherein T, X, W and Y are C; Z is O; R₁ is selected from aryl, arylalkyl, or heterocycle; R₆ is selected from arylalkenyl, arylalkyl, heterocycle, or heterocyclealkyl; and R₂, R₃, R₄, and R₅ are as defined in formula (I).

In another embodiment, compounds of the present invention have formula (I) wherein T, X, W and Y are C; Z is O; R₁ is heterocycle; R₆ is arylalkyl; and R₂, R₃, R₄, and R₅ are as defined in formula (I).

In another embodiment, compounds of the present invention have formula (I) wherein T, X, W and Y are C; Z is O; R₁ is heterocycle selected from furyl, pyridinyl, or pyrimidinyl, wherein the heterocycle is substituted with 0, 1, or 2 substituents selected from carboxy, haloalkyl, hydroxyalkyl, or (NR_{A}R_{B})carbonyl; R_{A} and R_{B} are independently selected from hydrogen, arylhydroxyalkyl, heterocyclealkyl, hydroxyalkyl, or (NZ₁Z₂)alkyl; R₂, R₃, R₄ and R₅ are hydrogen; R₆ is phenylmethyl; and Z₁ and Z₂ are as defined in formula (I).

In another embodiment, compounds of the present invention have formula (I) wherein T, X, W and Y are C; Z is O; R₁ is aryl; R₆ is arylalkyl; and R₂, R₃, R₄, and R₅ are as defined in formula (I).

In another embodiment, compounds of the present invention have formula (I) wherein T, X, W and Y are C; Z is O; R₁ is phenyl substituted with 0, 1, or 2 substituents selected from carboxy, heterocyclecarbonyl, or (NR_{A}R_{B})carbonyl; R_{A} and R_{B} are independently selected hydrogen, aryl, arylhydroxyalkyl, cycloalkyl, cycloalkylalkyl, heterocycle, heterocyclealkyl, hydroxyalkyl, or (NZ₁Z₂)alkyl; R₂, R₃, R₄ and R₅ are hydrogen; R₆ is phenylmethyl; and Z₁ and Z₂ are as defined in formula (I).

In another embodiment, compounds of the present invention have formula (I) wherein T, X, W and Y are C; Z is O; R₁ is arylalkyl; R₆ is arylalkenyl; and R₂, R₃, R₄, and R₅ are as defined in formula (I).

In another embodiment, compounds of the present invention have formula (I) wherein T, X, W and Y are C; Z is O; R₁ is phenylmethyl; R₂, R₃, R₄ and R₅ are hydrogen; and R₆ is arylalkenyl, wherein the aryl is phenyl.

In another embodiment, compounds of the present invention have formula (I) wherein T, X, W and Y are C; Z is O; R₁ is arylalkyl; R₆ is heterocyclealkyl; and R₂, R₃, R₄, and R₅ are as defined in formula (I).

In another embodiment, compounds of the present invention have formula (I) wherein T, X, W and Y are C; Z is O; R₁ is phenylmethyl; R₂, R₃, R₄ and R₅ are hydrogen; R₆ is furylmethyl wherein the furyl is substituted with 0, 1, or 2 substituents selected from carboxy, heterocyclecarbonyl, hydroxyalkyl, or (NR_{A}R_{B})carbonyl; R_{A} and R_{B} are independently selected from hydrogen, cycloalkyl, or (NZ₁Z₂)alkyl; Z₁ and Z₂ are as defined in formula (I).

In another embodiment, compounds of the present invention have formula (I) wherein T, X, and W are C; Y is N; Z is O; R₁ is selected from aryl, arylalkyl or heterocycle; R₅ is absent; R₆ is selected from arylalkenyl, arylalkyl, heterocycle, or heterocyclealkyl; and R₂, R₃, or R₄ are as defined in formula (I).

In another embodiment, compounds of the present invention have formula (I) wherein T, X, and W are C; Y is N; Z is O; R₁ is heterocycle; R₅ is absent; R₆ is arylalkyl; and R₂, R₃, or R₄ are as defined in formula (I).

In another embodiment, compounds of the present invention have formula (I) wherein T, X, and W are C; Y is N; Z is O; R₁ is heterocycle selected from furyl, pyridinyl or pyrimidinyl wherein the heterocycle is substituted with 0, 1 or 2 substituents selected from carboxy, haloalkyl, hydroxyalkyl or (NR_{A}R_{B})carbonyl wherein R_{A} and R_{B} are independently selected from the group consisting of hydrogen, arylhydroxyalkyl, heterocyclealkyl, hydroxyalkyl or (NZ₁Z₂)alkyl; R₂, R₃, and R₄ are hydrogen; R₅ is absent; and R₆ is 2-fluorophenylmethyl.

In another embodiment, compounds of the present invention have formula (I) wherein T, X, and W are C; Y is N; Z is O; R₁ is pyridinyl substituted with nitro; R₂, R₃, and R₄ are hydrogen; R₅ is absent; and R₆ is 2-fluorophenylmethyl.

In another embodiment, the present invention relates to a method of treating disorders that are ameliorated or prevented by increasing cGMP levels in a mammal comprising administering to the mammal a therapeutically effective amount of a compound of formula (I). The method provides for treatment of disorders such as sexual dysfunction, cardiovascular disease, atherosclerosis, angina pectoris, diastolic dysfunction, benign prostatic hyperplasia (BPH), incontinence, endothelial dysfunction, thrombosis, diabetes, liver cirhosis, cognitive disorders, Alzheimer's disease, anxiety, stress, depression, sleep disorders, migraine, cerebral ischemia, brain trauma, pain, memory and learning disorders. In particular, the method provides treatment for male erectile dysfunction.

In another embodiment, the present invention relates to a method of treating disorders that are ameliorated by increasing cGMP levels in a mammal comprising administering to the mammal a therapeutically effective amount of a compound of formula (I) in combination with a pharmaceutically acceptable carrier. The method provides for treatment of disorders such as sexual dysfunction, cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory and learning disorders. In particular, the method provides treatment for male erectile dysfunction.

In another embodiment, the present invention relates to a method of treating disorders that are ameliorated by increasing cGMP levels in a mammal comprising administering to the mammal a therapeutically effective amount of a compound of formula (I) in combination with a phosphodiesterase 5 inhibitor including, but not limited to, sildenafil or vardenafil. The method provides for treatment of disorders such as sexual dysfunction, cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory and learning disorders. In particular, the method provides treatment for male erectile dysfunction.

In another embodiment, the present invention relates to a method of treating disorders that are ameliorated by increasing cGMP levels in a mammal comprising administering to the mammal a therapeutically effective amount of a compound of formula (I) in combination with an adrenergic receptor antagonist including, but not limited to, terazosin, prazosin or tamsulosin. The method provides for treatment of disorders such as sexual dysfunction, cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory and learning disorders. In particular, the method provides treatment for male erectile dysfunction.

In another embodiment, the present invention relates to a method of treating disorders that are ameliorated by increasing cGMP levels in a mammal comprising administering to the mammal a therapeutically effective amount of a compound of formula (I) in combination with a dopamine receptor agonist including, but not limited to, apomorphine. The method provides for treatment of disorders such as sexual dysfunction, cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory and learning disorders. In particular, the method provides treatment for male erectile dysfunction.

### DEFINITIONS OF THE PRESENT INVENTION

As used throughout this specification and the appended claims, the following terms have the following meanings:

The term “alkenyl,” as used herein, refers to a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkoxy,” as used herein, refers to an alkoxy group, as defined herein, appended to the parent molecular moiety through another alkoxy group, as defined herein. Representative examples of alkoxyalkoxy include, but are not limited to, tert-butoxymethoxy, 2-ethoxyethoxy, 2-methoxyethoxy, and methoxymethoxy.

The term “alkoxyalkyl,” as used herein, refers to an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl,” as used herein, refers to an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkyl,” as used herein, refers to an alkoxycarbonyl group, as defined herein, appended to the parent molecular moiety through an alkyl group, as defined herein. Representative examples of alkoxycarbonylalkyl include, but are not limited to, 3-methoxycarbonylpropyl, 4-ethoxycarbonylbutyl, and 2-tert-butoxycarbonylethyl.

The term “alkoxysulfonyl,” as used herein, refers to an alkoxy group, as defined herein, appended appended to the parent molecular moiety through a sulfonyl group, as defined herein. Representative examples of alkoxysulfonyl include, but are not limited to, methoxysulfonyl, ethoxysulfonyl and propoxysulfonyl.

The term “alkyl,” as used herein, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl,” as used herein, refers to an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonylalkyl,” as used herein, refers to an alkylcarbonyl group, as defined herein, appended to the parent molecular moiety through an alkyl group, as defined herein. Representative examples of alkylcarbonylalkyl include, but are not limited to, 2-oxopropyl, 3,3-dimethyl-2-oxopropyl, 3-oxobutyl, and 3-oxopentyl.

The term “alkylcarbonyloxy,” as used herein, refers to an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkylene,” denotes a divalent group derived from a straight or branched chain hydrocarbon of from 1 to 10 carbon atoms. Representative examples of alkylene include, but are not limited to, —CH₂—, —CH₂CH₂—, —CH₂CH₂CH₂—, —CH₂CH₂CH₂CH₂—, and —CH₂CH(CH₃)CH₂—.

The term “alkylsulfinyl,” as used herein, refers to an alkyl group, as defined herein, appended to the parent molecular moiety through a sulfinyl group, as defined herein. Representative examples of alkylsulfinyl include, but are not limited to, methylsulfinyl and ethylsulfinyl.

The term “alkylsulfonyl,” as used herein, refers to an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylthio,” as used herein, refers to an alkyl group, as defined herein, appended to the parent molecular moiety through a sulfur atom. Representative examples of alkylthio include, but are not limited, methylsulfanyl, ethylsulfanyl, tert-butylsulfanyl, and hexylsulfanyl.

The term “alkylthioalkyl,” as used herein, refers to an alkylthio group, as defined herein, appended to the parent molecular moiety through an alkyl group, as defined herein. Representative examples of alkylthioalkyl include, but are not limited, methylsulfanylmethyl and 2-(ethylsulfanyl)ethyl.

The term “alkynyl,” as used herein, refers to a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, refers to a phenyl group, or a bicyclic or a tricyclic fused ring system wherein one or more of the fused rings is a phenyl group. Bicyclic fused ring systems are exemplified by a phenyl group fused to a cycloalkyl group, as defined herein, or another phenyl group. Tricyclic fused ring systems are exemplified by a bicyclic fused ring system, as defined herein, fused to a cycloalkyl group, as defined herein, or another phenyl group. Representative examples of aryl include, but are not limited to, anthracenyl, azulenyl, fluorenyl, 2,3-dihydroindenyl, indenyl, naphthyl, phenyl and tetrahydronaphthyl.

The aryl groups of this invention are substituted with 0, 1, 2, 3, 4 or 5 substituents independently selected from alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfonyl, alkylthio, alkylthioalkyl, alkynyl, arylalkyl, arylcarbonyl, carboxy, carboxyalkyl, cycloalkyl, cycloalkylalkyl, cyano, cyanoalkyl, ethylenedioxy, formyl, haloalkoxy, haloalkyl, halogen, heterocyclealkyl, heterocyclecarbonyl, hydroxy, hydroxyalkyl, mercapto, methylenedioxy, nitro, (NZ₁Z₂)alkyl, —NR_{A}R_{B}, (NR_{A}R_{B})carbonyl and (NR_{A}R_{B})sulfonyl. The aryl groups of this invention may also be substituted with 1 additional aryl group or 1 additional heterocycle wherein the additional aryl group and the additional heterocycle are substituted with 0, 1, 2, 3, 4 or 5 substituents independently selected from alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfonyl, alkylthio, alkylthioalkyl, alkynyl, carboxy, carboxyalkyl, cyano, cyanoalkyl, ethylenedioxy, formyl, haloalkoxy, haloalkyl, halogen, hydroxy, hydroxyalkyl, mercapto, methylenedioxy, nitro, (NZ₁Z₂)alkyl, —NR_{A}R_{B}, (NR_{A}R_{B})carbonyl and (NR_{A}R_{B})sulfonyl. Representative examples of aryl substituted with 0, 1, 2, 3, 4 or 5 substituents include, but are not limited to, 2-carboxyphenyl, 3-carboxyphenyl, 4-carboxyphenyl, 2-({4-[3-(dimethylamino)propyl]-1-piperazinyl}carbonyl)phenyl, 2-({4-[3-(dimethylamino)propyl]-1-piperazinyl}carbonyl)phenyl, 2-({4-[2-(dimethylamino)ethyl]-1-piperazinyl}carbonyl)phenyl, 2-{[(4-hydroxycyclohexyl)amino]carbonyl}phenyl, 2-({[(1R,2S)-1-hydroxy-2,3-dihydro-1H-inden-2-yl]amino}carbonyl)phenyl, 2-({[1-(hydroxymethyl)butyl]amino}carbonyl)phenyl, 2-{[(2-hydroxy-2-phenylethyl)amino]carbonyl}phenyl, ({[2-(1-methyl-2-pyrrolidinyl)ethyl]amino}carbonyl)phenyl, 2-({[3-(1H-imidazol-1-yl)propyl]amino}carbonyl)phenyl, 2-[(4-morpholinylamino)carbonyl]phenyl, 2-[({[(1S,2R)-2-hydroxycyclohexyl]methyl}amino)carbonyl]phenyl, and (1S)-1-hydroxy-2,3-dihydro-1H-inden-2-yl.

The term “arylalkenyl” as used herein, means an aryl group, as defined herein, appended to the parent molecular moiety through an alkenyl group, as defined herein. Representative examples of arylalkenyl include, but are not limited to, 3-phenyl-2-propenyl, 2-phenylvinyl, and 4-phenyl-3-butenyl.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and,2-naphth-2-ylethyl.

The term “arylcarbonyl” as used herein, means an aryl group, as defined herein, appended to the parent molecular moiety through a carbonyl group, as defined herein. Representative examples of arylcarbonyl include, but are not limited to, benzoyl, 2-chlorobenzoyl, 3-methylbenzoyl, 3,4-dichlorobenzoyl, and 2,4 difluorobenzoyl.

The term “arylhydroxyalkyl” as used herein, means an aryl group, as defined herein, appended to the parent molecular moiety through an hydroxyalkyl group, as defined herein. Representative examples of arylhydroxyalkyl include, but are not limited to, 2-hydroxy-2-phenylethyl, 2-hydroxy-3-phenylpropyl, and 3-hydroxy-3-phenylpropyl.

The term “carbonyl,” as used herein, refers to a —C(O)— group.

The term “carboxy,” as used herein, refers to a —CO₂H group.

The term “carboxyalkyl,” as used herein, refers to a carboxy group, as defined herein, appended to the parent molecular moiety through an alkyl group, as defined herein. Representative examples of carboxyalkyl include, but are not limited to, carboxymethyl, 2-carboxyethyl, and 3-carboxypropyl.

The term “cyano,” as used herein, refers to a —CN group.

The term “cyanoalkyl,” as used herein, refers to a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “cycloalkyl” as used herein, means a saturated cyclic hydrocarbon group containing from 3 to 8 carbons. Examples of cycloalkyl include cyclopropyl, cyclobutyl, cyclopentyl, cyclohexyl, cycloheptyl, and cyclooctyl.

The cycloalkyl groups of this invention are substituted with 0, 1, 2 or 3 substituents independently selected from alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfonyl, alkylthio, alkylthioalkyl, alkynyl, carboxy, carboxyalkyl, cyano, cyanoalkyl, formyl, haloalkoxy, haloalkyl, halogen, hydroxy, hydroxyalkyl, mercapto, oxo, (NZ₁Z₂)alkyl, —NR_{A}R_{B}, (NR_{A}R_{B})carbonyl and (NR_{A}R_{B})sulfonyl. Representative examples of cycloalkyl substituted with 0, 1, 2 or 3 substituents include, but are not limited to, 4-hydroxycyclohexyl and 2-hydroxycyclohexyl.

The term “ethylenedioxy,” as used herein, refers to a —O(CH₂)₂O— group wherein the oxygen atoms of the ethylenedioxy group are attached to the parent molecular moiety through one carbon atom forming a 5 membered ring or the oxygen atoms of the ethylenedioxy group are attached to the parent molecular moiety through two adjacent carbon atoms forming a six membered ring.

The term “formyl,” as used herein, refers to a —C(O)H group.

The term “halo” or “halogen,” as used herein, refers to —Cl, —Br, —I or —F.

The term “haloalkoxy,” as used herein, refers to at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl,” as used herein, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heterocycle” or “heterocyclic,” as used herein, refers to a monocyclic, bicyclic, or tricyclic ring system. Monocyclic ring systems are exemplified by any 3- or 4-membered ring containing a heteroatom independently selected from oxygen, nitrogen and sulfur; or a 5-, 6- or 7-membered ring containing one, two or three heteroatoms wherein the heteroatoms are independently selected from nitrogen, oxygen and sulfur. The 5-membered ring has from 0-2 double bonds and the 6- and 7-membered ring have from 0-3 double bonds. Representative examples of monocyclic ring systems include, but are not limited to, azetidinyl, azepanyl, aziridinyl, diazepinyl, 1,3-dioxolanyl, dioxanyl, dithianyl, furyl, imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinyl, pyranyl, pyrazinyl, pyrazolyl, pyrazolinyl, pyrazolidinyl, pyridinyl, pyrimidinyl, pyridazinyl, pyrrolyl, pyrrolinyl, pyrrolidinyl, tetrahydrofuranyl, tetrahydrothienyl, tetrazinyl, tetrazolyl, thiadiazolyl, thiadiazolinyl, thiadiazolidinyl, thiazolyl, thiazolinyl, thiazolidinyl, thienyl, thiomorpholinyl, 1,1-dioxidothiomorpholinyl (thiomorpholine sulfone), thiopyranyl, triazinyl, triazolyl, and trithianyl. Bicyclic ring systems are exemplified by any of the above monocyclic ring systems fused to an aryl group as defined herein, a cycloalkyl group as defined herein, or another monocyclic ring system. Representative examples of bicyclic ring systems include but are not limited to, for example, benzimidazolyl, benzodioxinyl, benzothiazolyl, benzothienyl, benzotriazolyl, benzoxazolyl, benzofuranyl, benzopyranyl, benzothiopyranyl, cinnolinyl, indazolyl, indolyl, 2,3-dihydroindolyl, indolizinyl, naphthyridinyl, isobenzofuranyl, isobenzothienyl, isoindolyl, isoquinolinyl, phthalazinyl, pyranopyridinyl, quinolinyl, quinolizinyl, quinoxalinyl, quinazolinyl, tetrahydroisoquinolinyl, tetrahydroquinolinyl, and thiopyranopyridinyl. Tricyclic rings systems are exemplified by any of the above bicyclic ring systems fused to an aryl group as defined herein, a cycloalkyl group as defined herein, or a monocyclic ring system. Representative examples of tricyclic ring systems include, but are not limited to, acridinyl, carbazolyl, carbolinyl, dibenzo[b,d]furanyl, dibenzo[b,d]thienyl, naphtho[2,3-b]furan, naphtho[2,3-b]thienyl, phenazinyl, phenothiazinyl, phenoxazinyl, thianthrenyl, thioxanthenyl and xanthenyl.

The heterocycles of this invention are substituted with 0, 1, 2, or 3 substituents independently selected from alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfonyl, alkylthio, alkylthioalkyl, alkynyl, carboxy, carboxyalkyl, cyano, cyanoalkyl, ethylenedioxy, formyl, haloalkoxy, haloalkyl, halogen, hydroxy, hydroxyalkyl, mercapto, methylenedioxy, nitro, oxo, (NZ₁Z₂)alkyl, —NR_{A}R_{B}, (NR_{A}R_{B})carbonyl and (NR_{A}R_{B})sulfonyl. The heterocycle groups of this invention may also be substituted with 1 additional aryl group or 1 additional heterocycle wherein the additional aryl group and the additional heterocycle are substituted with 0, 1, 2, 3, 4 or 5 substituents independently selected from alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfonyl, alkylthio, alkylthioalkyl, alkynyl, carboxy, carboxyalkyl, cyano, cyanoalkyl, ethylenedioxy, formyl, haloalkoxy, haloalkyl, halogen, hydroxy, hydroxyalkyl, mercapto, methylenedioxy, nitro, (NZ₁Z₂)alkyl, —NR_{A}R_{B}, (NR_{A}R_{B})carbonyl and (NR_{A}R_{B})sulfonyl.

Representative examples of heterocycle substituted with 0, 1, 2, or 3 substituents include, but are not limited to, 5-hydroxymethyl-2-furyl, 5-carboxy-2-furyl, 6-hydroxymethyl-2-pyridinyl, 5-({[3-(dimethylamino)propyl]amino}carbonyl)-2-furyl, 5-{[(4-hydroxybutyl)amino]carbonyl}-2-furyl, 5-trifluoromethyl-3-pyridinyl, 3-(aminocarbonyl)-2-pyridinyl, 5-({[2-(4-morpholinyl)ethyl]amino}carbonyl)-2-furyl, 5-{[(2-hydroxy-2-phenylethyl)amino]carbonyl}-2-furyl, 5-{[(5-hydroxy-1,5-dimethylhexyl)amino]carbonyl}-2-furyl, 5-({[1-(hydroxymethyl)butyl]amino}carbonyl)-2-furyl, 5-({[(4-methyl-1-piperazinyl)carbonyl]amino}carbonyl)-2-furyl, 5-{[(2R)-2-(hydroxymethyl)-1-pyrrolidinyl]carbonyl}-2-furyl, 5-{[(2S)-2-(hydroxymethyl)-1-pyrrolidinyl]carbonyl}-2-furyl, and 5-[(4-hydroxy-1-piperidinyl)carbonyl]-2-furyl.

The term “heterocyclealkenyl” as used herein, means a heterocycle, as defined herein, appended to the parent molecular moiety through an alkenyl group, as defined herein. Representative examples of heterocyclealkenyl include, but are not limited to, 3-pyridin-3-yl-2-propenyl, and 4-pyrimidin-2-yl-3-butenyl.

The term “heterocyclealkyl” as used herein, means a heterocycle, as defined herein, appended to the parent molecular moiety through an alkyl group, as defined herein. Representative examples of heterocyclealkyl include, but are not limited to, pyridin-3-ylmethyl, 2-pyrimidin-2-ylpropyl, 2-(4-morpholinyl)ethyl, and 3-(1H-imidazol-1H-yl)propyl.

The term “heterocyclecarbonyl” as used herein, means a heterocycle, as defined herein, appended to the parent molecular moiety through a carbonyl group, as defined herein. Representative examples of heterocyclecarbonyl include, but are not limited to, 3-pyridinylcarbonyl, 4-morpholinylcarbonyl, 1-piperazinylcarbonyl, and {4-[3-(dimethylamino)propyl]-1-piperazinyl}carbonyl.

The term “hydroxy,” as used herein, refers to an —OH group.

The term “hydroxyalkyl,” as used herein, refers to at least one hydroxy group, as defined herein, appended to the parent molecular moiety through an alkyl group, as defined herein. Representative examples of hydroxyalkyl include, but are not limited to, hydroxymethyl, 2-hydroxyethyl, 3-hydroxypropyl, 2-ethyl-4-hydroxyheptyl and 2,3-dihydroxypropyl.

The term “mercapto,” as used herein, refers to a —SH group.

The term “methylenedioxy,” as used herein, refers to a —OCH₂O— group wherein the oxygen atoms of the methylenedioxy are attached to the parent molecular moiety through two adjacent carbon atoms.

The term “nitro,” as used herein, refers to a —NO₂ group.

The term “—NR_{A}R_{B},” as used herein, refers to two groups, R_{A} and R_{B}, which are appended to the parent molecular moiety through a nitrogen atom. R_{A} and R_{B} are each independently selected from hydrogen, alkyl, alkylcarbonyl, aryl, arylalkyl, arylhydroxyalkyl, cycloalkyl, cycloalkylalkyl, formyl, heterocycle, heterocyclealkyl, hydroxyalkyl, or (NZ₁Z₂)alkyl. Representative examples of —NR_{A}R_{B} include, but are not limited to, amino, methylamino, dimethylamino, (2-(dimethylamino)ethyl)amino, (3-(dimethylamino)propyl)amino, (4-hydroxybutyl)amino, (1-(hydroxymethyl)butyl)amino, (5-hydroxy-1,5-dimethylhexyl)amino, (2-hydroxy-2-phenylethyl)amino, (2-(4-morpholinyl)ethyl)amino, (4-hydroxycyclohexyl)amino, ((1S)-1-hydroxy-2,3-dihydro-1H-inden-2-yl)amino, (2-(1-methyl-2-pyrrolidinyl)ethyl)amino, (3-(1H-imidazol-1-yl)propyl)amino, (4-morpholinyl)amino, and ((1S,2R)-2-hydroxycyclohexylmethyl)amino.

The term “(NR_{A}R_{B})carbonyl,” as used herein, refers to a —NR_{A}R_{B} group, as defined herein, appended to the parent molecular moiety through a carbonyl group, as defined herein. Representative examples of (NR_{A}R_{B})carbonyl include, but are not limited to, aminocarbonyl, (methylamino)carbonyl, (dimethylamino)carbonyl, (ethylmethylamino)carbonyl, (3-(dimethylamino)propyl)aminocarbonyl, (2-(dimethylamino)ethyl)aminocarbonyl, (5-hydroxy-1,5-dimethylhexyl)aminocarbonyl, (4-hydroxybutyl)aminocarbonyl and (1-(hydroxymethyl)butyl)aminocarbonyl.

The term “(NR_{A}R_{B})sulfonyl,” as used herein, refers to a —NR_{A}R_{B} group, as defined herein, appended to the parent molecular moiety through a sulfonyl group, as defined herein. Representative examples of (NR_{A}R_{B})sulfonyl include, but are not limited to, aminosulfonyl, (methylamino)sulfonyl, (dimethylamino)sulfonyl, (ethylmethylamino)sulfonyl, (3-(dimethylamino)propyl)aminosulfonyl, and (2-(dimethylamino)ethyl)aminosulfonyl.

The term “—NZ₁Z₂,” as used herein, refers to two groups, Z₁ and Z₂, which are appended to the parent molecular moiety through a nitrogen atom. Z₁ and Z₂ are each independently selected from hydrogen, alkyl, alkylcarbonyl and formyl. Representative examples of —NZ₁Z₂ include, but are not limited to, amino, methylamino, acetylamino, and acetylmethylamino.

The term “(NZ₁Z₂)alkyl,” as used herein, refers to a —NZ₁Z₂ group, as defined herein, appended to the parent molecular moiety through an alkyl group, as defined herein. Representative examples of (NZ₁Z₂)alkyl include, but are not limited to, 2-(amino)ethyl, 2-(dimethylamino)ethyl, 3-(amino)propyl, 3-(dimethylamino)propyl and (ethylmethylamino)carbonyl.

The term “(NZ₁Z₂)carbonyl,” as used herein, refers to a —NZ₁Z₂ group, as defined herein, appended to the parent molecular moiety through a carbonyl group, as defined herein. Representative examples of (NZ₁Z₂)carbonyl include, but are not limited to, aminocarbonyl, (methylamino)carbonyl, (dimethylamino)carbonyl and (ethylmethylamino)carbonyl.

The term “(NZ₁Z₂)sulfonyl,” as used herein, refers to a —NZ₁Z₂ group, as defined herein, appended to the parent molecular moiety through a sulfonyl group, as defined herein. Representative examples of (NZ₁Z₂)sulfonyl include, but are not limited to, aminosulfonyl, (methylamino)sulfonyl, (dimethylamino)sulfonyl and (ethylmethylamino)sulfonyl.

The term “oxo,” as used herein, refers to a ═0 moiety.

The term “sulfinyl,” as used herein, refers to a —S(O)— group.

The term “sulfonyl,” as used herein, refers to a —SO₂— group.

The term “sexual dysfunction,” as used herein refers to male sexual dysfunction and female sexual dysfunction.

The term “male sexual dysfunction,” as used herein includes, but is not limited to, male erectile dysfunction and premature ejacualtion.

The term “female sexual dysfunction,” as used herein includes, but is not limited to, female anorgasmia, clitoral erectile insufficiency, vaginal engorgement, dyspareunia, and vaginismus.

Compounds of the present invention may exist as stereoisomers wherein, asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, Pure Appl. Chem., 1976, 45: 13-30. The present invention contemplates various stereoisomers and mixtures thereof and are specifically included within the scope of this invention. Stereoisomers include enantiomers and diastereomers, and mixtures of enantiomers or diastereomers. Individual stereoisomers of compounds of the present invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

Representative compounds of the present invention include:

{5-[(1-benzyl-1H-indazol-3-yl)oxy]-2-furyl}methanol;

{6-[(1-benzyl-1H-indazol-3-yl)oxy]-2-pyridinyl}methanol;

5-[(1-benzyl-1H-indazol-3-yl)oxy]-2-furaldehyde (6-chloro-3-pyridazinyl)hydrazone;

5-[(1-benzyl-1H-indazol-3-yl)oxy]-2-furoic acid;

5-[(1-benzyl-1H-indazol-3-yl)oxy]-N-[3-(dimethylamino)propyl]-2-furamide;

5-[(1-benzyl-1H-indazol-3-yl)oxy]-N-(4-hydroxybutyl)-2-furamide;

5-[(1-benzyl-1H-indazol-3-yl)oxy]-N-[1-(hydroxymethyl)butyl]-2-furamide;

5-[(1-benzyl-1H-indazol-3-yl)oxy]-N-(5-hydroxy-1,5-dimethylhexyl)-2-furamide;

5-[(1-benzyl-1H-indazol-3-yl)oxy]-N-(2-hydroxy-2-phenylethyl)-2-furamide;

5-[(1-benzyl-1H-indazol-3-yl)oxy]-N-[2-(4-morpholinyl)ethyl]-2-furamide;

1-benzyl-3-(2-pyrimidinyloxy)-1H-indazole;

1-benzyl-3-{[5-(trifluoromethyl)-3-pyridinyl]oxy}-1H-indazole;

2-[(1-benzyl-1H-indazol-3-yl)oxy]nicotinamide;

2-[(1-benzyl-1H-indazol-3-yl)oxy]benzoic acid;

2-[(1-benzyl-1H-indazol-3-yl)oxy]-N-[2-(dimethylamino)ethyl]benzamide;

N-[3-(4-{2-[(1-benzyl-1H-indazol-3-yl)oxy]benzoyl}-1-piperazinyl)propyl]-N,N-dimethylamine;

2-[(1-benzyl-1H-indazol-3-yl)oxy]-N-[3-(dimethylamino)propyl]benzamide;

2-[(1-benzyl-1H-indazol-3-yl)oxy]-N-(4-hydroxycyclohexyl)benzamide;

2-[(1-benzyl-1H-indazol-3-yl)oxy]-N-[(1R,2S)-1-hydroxy-2,3-dihydro-1H-inden-2-yl]benzamide;

2-[(1-benzyl-1H-indazol-3-yl)oxy]-N-[1-(hydroxymethyl)butyl]benzamide;

2-[(1-benzyl-1H-indazol-3-yl)oxy]-N-(2-hydroxy-2-phenylethyl)benzamide;

2-[(1-benzyl-1H-indazol-3-yl)oxy]-N-[2-(1-methyl-2-pyrrolidinyl)ethyl]benzamide;

2-[(1-benzyl-1H-indazol-3-yl)oxy]-N-[3-(1H-imidazol-1-yl)propyl]benzamide;

2-[(1-benzyl-1H-indazol-3-yl)oxy]-N′-(4-morpholinyl)benzohydrazide;

2-[(1-benzyl-1H-indazol-3-yl)oxy]-N-[(2-hydroxycyclohexyl)methyl]benzamide;

3-(benzyloxy)-1-[3-phenyl-2-propenyl]-1H-indazole;

5-{[3-(benzyloxy)-1H-indazol-1-yl]methyl}-2-furoic acid;

5-{[3-(benzyloxy)-1H-indazol-1-yl]methyl}-2-furyl)methanol;

3-(benzyloxy)-1-({5-[(4-methyl-1-piperazinyl)carbonyl]-2-furyl}methyl)-1H-indazole;

[1-(5-{[3-(benzyloxy)-1H-indazol-1-yl]methyl}-2-furoyl)-2-pyrrolidinyl]methanol;

5-{[3-(benzyloxy)-1H-indazol-1-yl]methyl}-N-[3-(dimethylamino)propyl]-2-furamide; and

5-{[3-(benzyloxy)-1H-indazol-1-yl]methyl}-N-(4-hydroxycyclohexyl)-2-furamide, or a pharmaceutically acceptable salt, ester, amide or prodrug thereof.

A preferred compound of the present invention is 1-benzyl-3-(2-pyridinyloxy)-1H-indazole or a pharmaceutically acceptable salt, ester, amide or prodrug thereof.

### ABBREVIATIONS

Abbreviations which have been used in the descriptions of the Schemes and the Examples that follow are: Boc for tert-butoxycarbonyl; DMF for N,N-dimethylformamide; DMSO for dimethylsulfoxide; EDCI or EDC for 1-ethyl-3-[3-(dimethylamino)propyl]-carbodiimide hydrochloride; Et for ethyl; EtOAc for ethyl acetate; EtOH for ethanol; HObt for 1-hydroxybenzotriazole; MeOH for methanol; TEA for triethylamine; TFA for trifluoroacetic acid; THF for tetrahydrofuran; TLC for thin layer chromatography; Tos for (4-methylphenyl)sulfonyl.

### PREPARATION OF COMPOUNDS OF THE PRESENT INVENTION

The compounds of the present invention may be prepared by a variety of synthetic routes. Representative procedures are described in Schemes 1-13.

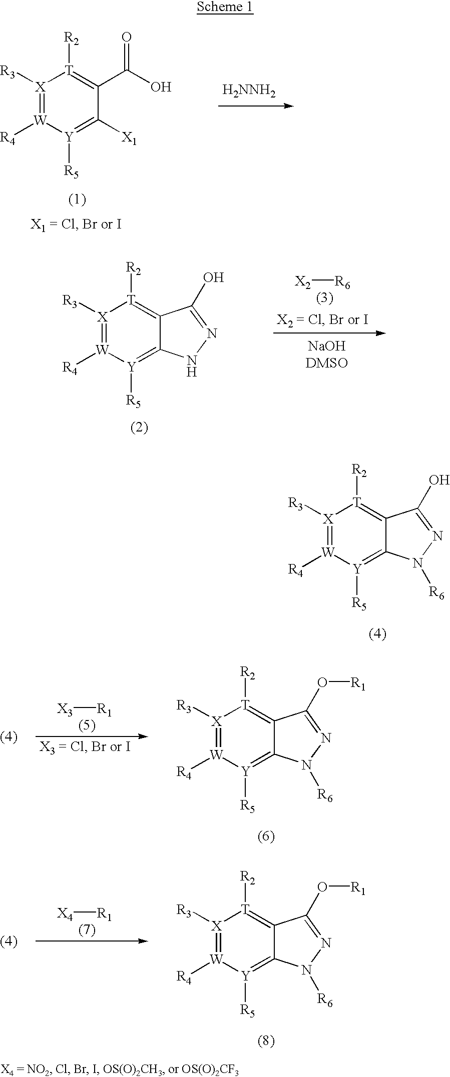

Indazoles of general formula (6), wherein T, X, W, Y, R₁, R₂, R₃, R₄, R₅, and R₆ are as defined in formula (I) can be prepared as described in Scheme 1. Benzoic acids of general formula (1) can be treated with hydrazine as described in L. Baiocchi et al., Synthesis, (1978) 633-648; V. J. Aran et al., Liebigs Ann., (1996) 683-691; and G. Zoni et al., Boll. Chim. Farm., (1968) 107, 598-605; to provide hydroxy indazoles of general formula (2). Hydroxyindazoles of general formula (2) can be treated with compounds of general formula (3) in the presence of sodium hydroxide to provide 1-substituted 3-hydroxyindazoles of general formula (4). 1-Substituted 3-hydroxyindazoles of general formula (4) can be treated with compounds of general formula (5) in the presence of sodium hydroxide or potassium hydroxide to provide indazoles of general formula (6).

Indazoles of general formula (8), wherein R₁, R₂, R₃, R₄, R₅, and R₆ are as defined in formula (I), can be prepared as described in Scheme 1. 1-Substituted 3-hydroxyindazoles of general formula (4) can be treated with a base such as sodium hydride and aryl groups or heterocycles of general formula (7), wherein X₄ is selected from NO₂, Cl, Br, I, OS(O)₂CH₃ or OS(O)₂CF₃, to provide indazoles of general formula (8). Alternatively, 1-substituted 3-hydroxyindazoles of general formula (4) can be treated with a base such as potassium carbonate, CuI and aryl groups or heterocycles of general formula (7) to provide indazoles of general formula (8).

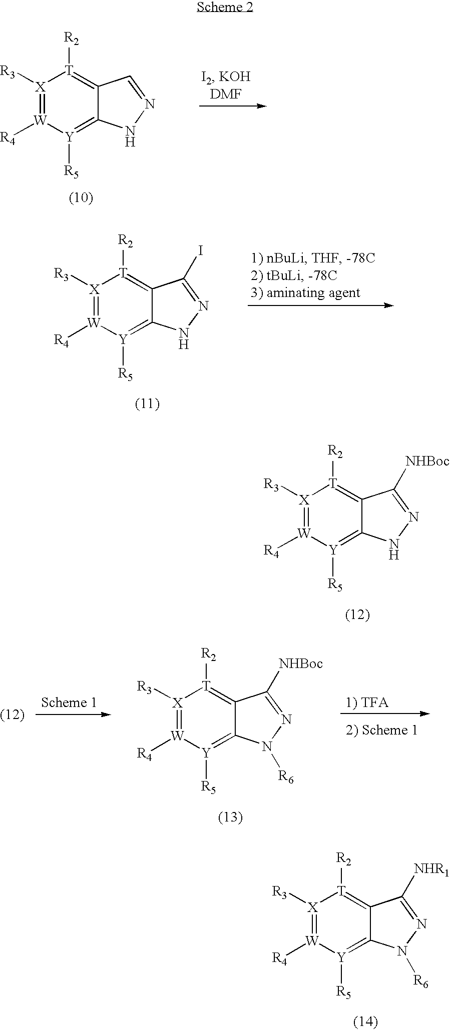

Indazoles of general formula (14), wherein T, X, W, Y, R₁, R₂, R₃, R₄, R₅, and R₆ are as defined in formula (I), can be prepared as described in Scheme 2. Indazoles of general formula (10), purchased or prepared as described in the Examples and Schemes contained herein or prepared as described in R. Huisgen, K. Bast, Organic Syntheses, Coll. Vol. V, 650-653 (1973); Ed. John Wiley and Sons, New York-London, Sydney-Toronto; J. J. Song, N. K. Yee, Tetrahedron Letters, 2937-2940 (2001); and Heterocyclic Compounds, Chapter 10, Indazole and condensed types, p. 289-382, Ed. R. H. Wiley, New York-London-Sydney, can be treated with iodine and a base such as potassium hydroxide to provide 3-iodoindazoles of general formula (11). 3-Iodoindazoles of general formula (11) can be treated with n-bultyllithium followed by treatment with tert-butyllithium as described in Welch, W. M., et al. Synthesis, (1992) 937-939 to provide the dianion which can be treated with an aminating agent such as BocN(Li)OTos, prepared as described in C. Greek, L. Bischoff, A. Girard, J. Hajicek, J-P. Genet, Bull. Soc. Chim. Fr., 131, 429-433 (1994), to provide 3-N-Bocindazoles of general formula (12) as described in Greek, C. et al., Bull. Soc. Chim. Fr., (1994) 131, 429-433. 3-N-Bocindazoles of general formula (12) can be processed as described in Scheme 1 to provide N-Boc-indazoles of general formula (13). N-Boc-indazoles of general formula (13) can be treated with trifluoroacetic acid and then treated as described in Scheme 1 to provide Indazoles of general formula (14).

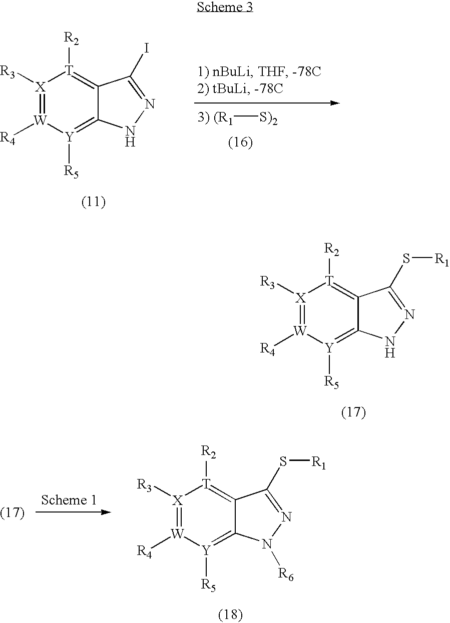

Indazoles of general formula (18), wherein T, X, W, Y, R₁, R₂, R₃, R₄, R₅, and R₆ are as defined in formula (I), can be prepared as described in Scheme 3. 3-Iodoindazoles of general formula (11) can be treated with n-bultyllithium followed by treatment with tert-butyllithium as described in Welch, W. M., et al. Synthesis, (1992) 937-939 to provide the dianion which can be treated with a disulfide of general formula (16) to provide indazoles of general formula (17) as described in Tetrahedron (2000) 56, 3709-3716. Indazoles of general formula (17) can be processed as described in Scheme 1 to provide indazoles of general formula (18).

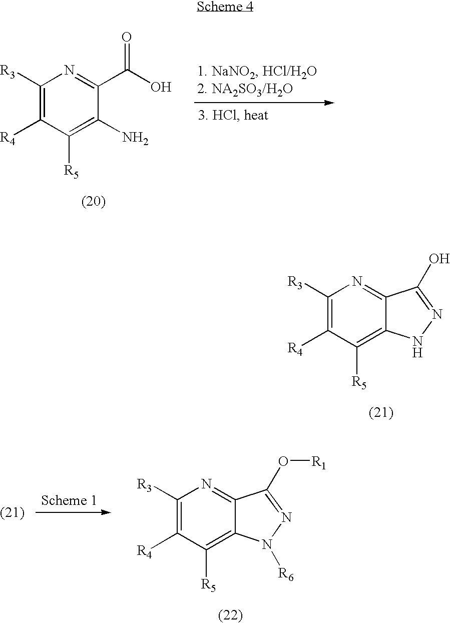

Pyrazolo[4,3-b]pyridines of general formula (22), wherein R₁, R₃, R₄, R₅, and R₆ are as defined in formula (I), can be prepared as described in Scheme 4. 3-Aminopyridine-2-carboxylic acids of general formula (20) can be treated with sodium nitrite and HCl/H₂O followed by treatment with Na₂SO₃/H₂O and then acidification with HCl to provide hydroxypyrazolo[4,3-b]pyridines of general formula (21) as described in J. Med. Chem. (1989) 32, 2128. Hydroxypyrazolo[4,3-b]pyridines of general formula (21) can be processed as described in Scheme 1 to provide pyrazolo[4,3-b]pyridines of general formula (22).

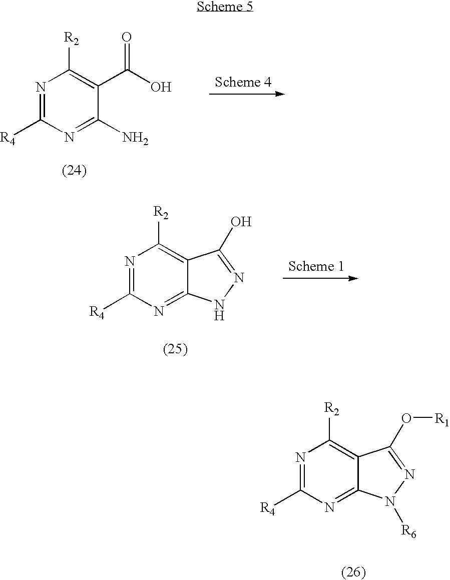

Pyrazolo[3,4-d]pyrimidines of general formula (26), wherein R₁, R₂, R₄, and R₆ are as defined in formula (I), can be prepared as described in Scheme 5. 4-Aminopyrimidine-5-carboxylic acids of general formula (24) can be processed as described in Scheme 4 to provide hydroxypyrazolo[3,4-d]pyrimidines of general formula (25). Hydroxypyrazolo[3,4-d]pyrimidines of general formula (25) can be processed as described in Scheme 1 to provide pyrazolo[3,4-d]pyrimidines of general formula (26).

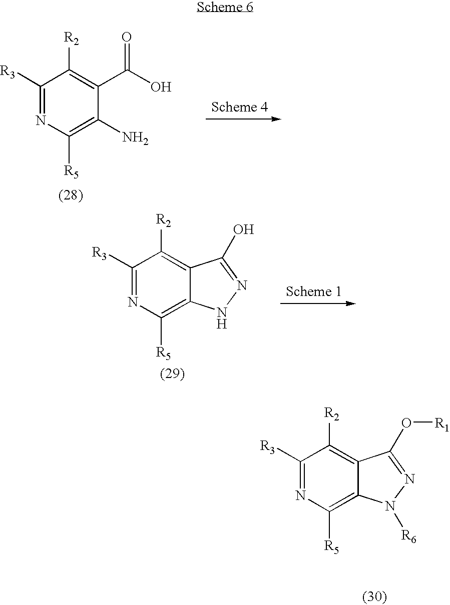

Pyrazolo[3,4-c]pyridines of general formula (30), wherein R₁, R₂, R₃, R₅, and R₆ are as defined in formula (I), can be prepared as described in Scheme 6. 3-Aminoisonicotinic acids of general formula (28), can be processed as described in Scheme 4 and Scheme 1 to provide pyrazolo[3,4-c]pyridines of general formula (30).

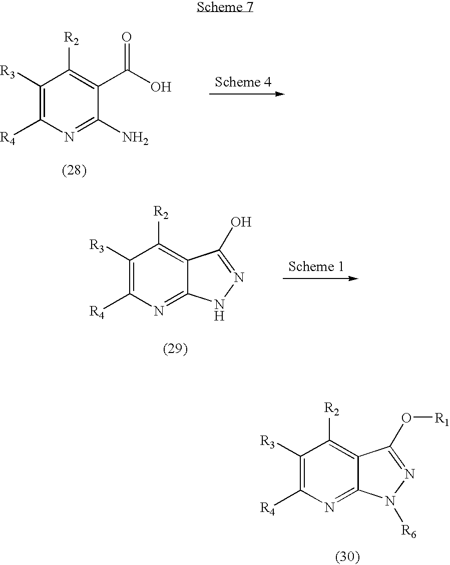

Pyrazolo[3,4-b]pyridines of general formula (28), wherein R₁, R₂, R₃, R₄, and R₆ are as defined in formula (I), can be prepared as described in Scheme 7. 2-Aminonicotinic acids of general formula (28) can be processed as described in Scheme 4 and Scheme 1 to provide pyrazolo[3,4-b]pyridines of general formula (28).

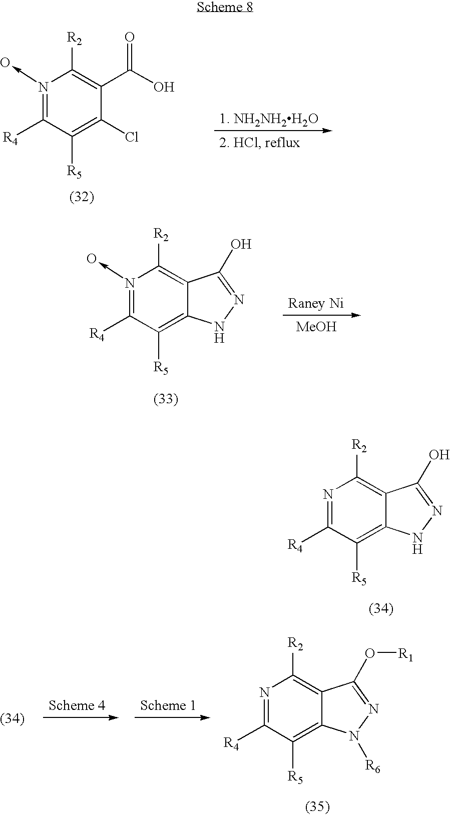

Pyrazolo[4,3-c]pyridines of general formula (35), R₁, R₂, R₄, R₅, and R₆ are as defined in formula (I), can be prepared as described in Scheme 8. N-Oxides of general formula (32) can be treated with hydrazine and then treated with refluxing hydrochloric acid to provide N-oxides of general formula (33). N-Oxides of general formula (33) can be reduced with Raney nickel as described in Aust. J. Chem., (1965) 18, 379-87 to provide hydroxypyrazolo[4,3-c]pyridines of general formula (34). Hydroxypyrazolo[4,3-c]pyridines of general formula (34) can be processed as described in Scheme 1 to provide pyrazolo[4,3-c]pyridines of general formula (35).

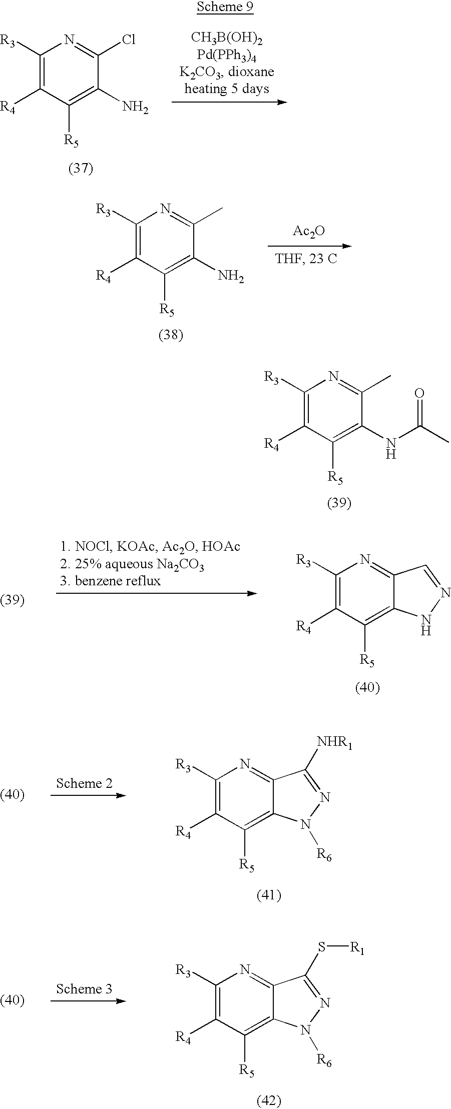

Pyrazolo[4,3-b]pyridines of general formula (41), wherein R₁, R₃, R₄, R₅, and R₆ are as defined in formula (I), can be prepared as described in Scheme 9. 2-Chloropyridin-3-amines of general formula (37) can be treated with methylboronic acid under Suzuki conditions as described in Tetrahedron (1998) 6311; JACS 1940, 2228 to provide aminopyridines of general formula (38). Aminopyridines of general formula (38) can be treated with acetic anhydride as described in J. Med. Chem. (1980) 848 to provide acetamides of general formula (39). Acetamides of general formula (39) can be treated with nitrosyl chloride to provide the nitroso compound which can be rearranged in refluxing benzene to pyrazolo[4,3-b]pyridines of general formula (40) as described in J.C.S. Perkin I (1973) 2901. Pyrazolo[4,3-b]pyridines of general formula (40) can be processed as described in Scheme 2 to provide Pyrazolo[4,3-b]pyridines of general formula (41).

Pyrazolo[4,3-b]pyridines of general formula (42), wherein R₁, R₃, R₄, R₅, and R₆ are as defined in formula (I), can be prepared as described in Scheme 9. Pyrazolo[4,3-b]pyridines of general formula (40) can be processed as described in Scheme 3 to provide Pyrazolo[4,3-b]pyridines of general formula (42).

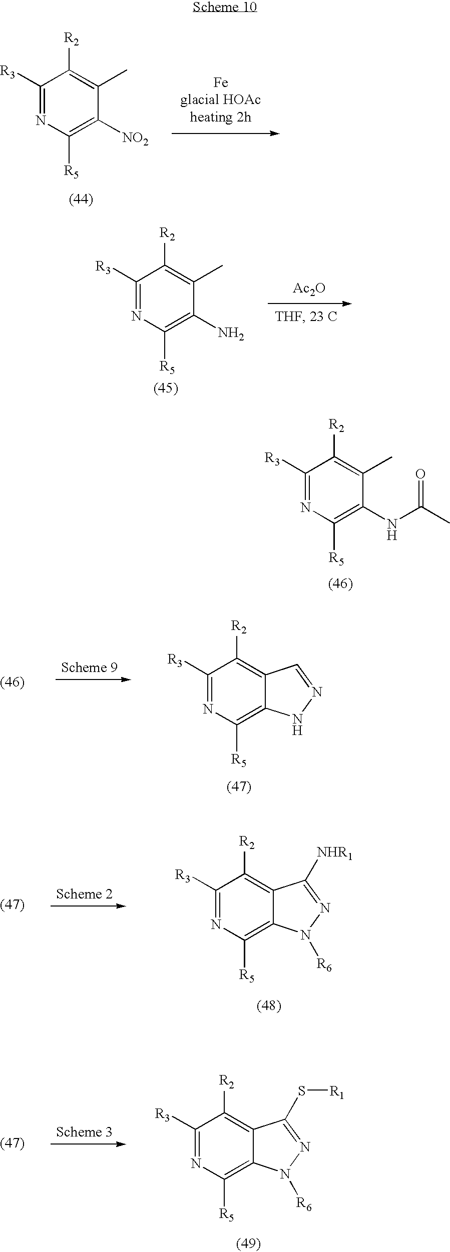

Pyrazolo[3,4-c]pyridines of general formula (48), wherein R₁, R₂, R₃, R₅, and R₆ are as defined in formula (I), can be prepared as described in Scheme 10. Nitropyridines of general formula (44) can be treated with iron in glacial acetic acid to provide aminopyridines of general formula (45) as described in Bull. Soc. Chim. Fr. (1992) 79. Aminopyridines of general formula (45) can be treated with acetic anhydride to provide acetamides of general formula (46) as described in J. Med. Chem. 1980, 23, 848. Acetamides of general formula (46) can be processed as described in Scheme 9 to provide pyrazolo[3,4-c]pyridines of general formula (47). Pyrazolo[3,4-c]pyridines of general formula (47) can be processed as described in Scheme 2 to provide pyrazolo[3,4-c]pyridines of general formula (48).

Pyrazolo[3,4-c]pyridines of general formula (49), wherein R₁, R₂, R₃, R₅, and R₆ are as defined in formula (I), can also be prepared as described in Scheme 10. Pyrazolo[3,4-c]pyridines of general formula (47) can be processed as described in Scheme 3 to provide pyrazolo[3,4-c]pyridines of general formula (49).

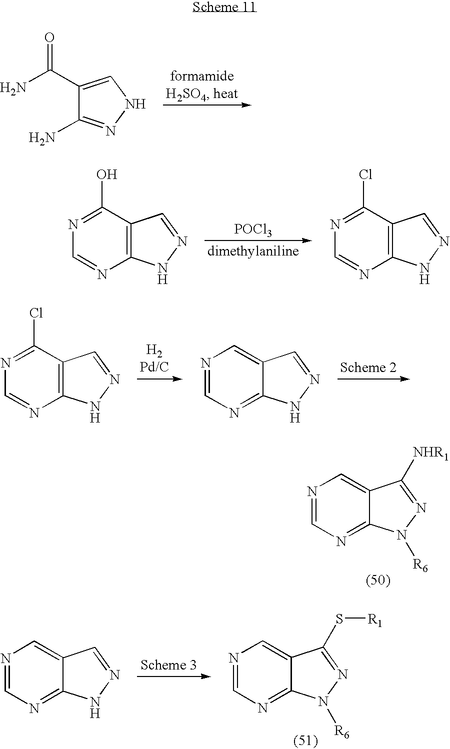

Pyrazolo[3,4-d]pyrimidines of general formula (50), wherein R₁ and R₆ are as defined in formula (I), can be prepared as described in Scheme 11. 3-Amino-1H-pyrazole-4-carboxamide can be heated at 185° C. with sulfuric acid in formamide to provide 1H-pyrazolo[3,4-d]pyrimidin-4-ol. 1H-Pyrazolo[3,4-d]pyrimidin-4-ol can be treated with phosphorous oxychloride and dimethylaniline to provide 4-chloro-1H-pyrazolo[3,4-d]pyrimidine. 4-Chloro-1H-pyrazolo[3,4-d]pyrimidine can be treated with a transition metal catalyst such as pallladium on carbon under a hydrogen atmosphere to provide pyrazolo[3,4-d]pyrimidine as described in JACS (1956) 784. Pyrazolo[3,4-d]pyrimidine can be processed as described in Scheme 2 to provide pyrazolo[3,4-d]pyrimidines of general formula (50).

Pyrazolo[3,4-d]pyrimidines of general formula (51), wherein R₁ and R₆ are as defined in formula (I), can also be prepared as described in Scheme 11. Pyrazolo[3,4-d]pyrimidine can be processed as described in Scheme 3 to provide pyrazolo[3,4-d]pyrimidines of general formula (51).

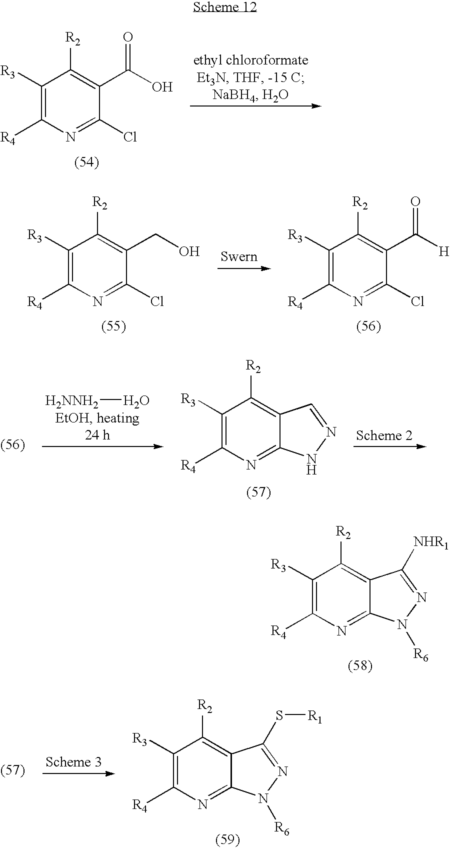

Pyrazolo[3,4-b]pyridines of general formula (58), wherein R₁, R₂, R₃, R₄, and R₆ are as defined in formula (I), can be prepared as described in Scheme 12. 2-Chloro-3-nicotinic acids of general formula (54) can be treated with ethyl chloroformate and sodium borohydride followed by Swern oxidation with dimethylsulfoxide, oxalyl chloride, and triethylamine to provide aldehydes of general formula (56) as described in Chem. Pharm. Bull. (2000) 694. Aldehydes of general formula (56) can be treated with hydrazine hydrate in refluxing ethanol to provide pyrazolo[3,4-b]pyridines of general formula (57) as described in Can. J. Chem. (1988) 420. Pyrazolo[3,4-b]pyridines of general formula (57) can be processed as described in Scheme 2 to provide pyrazolo[3,4-b]pyridines of general formula (58).

Pyrazolo[3,4-b]pyridines of general formula (59), wherein R₁, R₂, R₃, R₄, and R₆ are as defined in formula (I), can be prepared as described in Scheme 12. Pyrazolo[3,4-b]pyridines of general formula (57) can be processed as described in Scheme 3 to provide pyrazolo[3,4-b]pyridines of general formula (59).

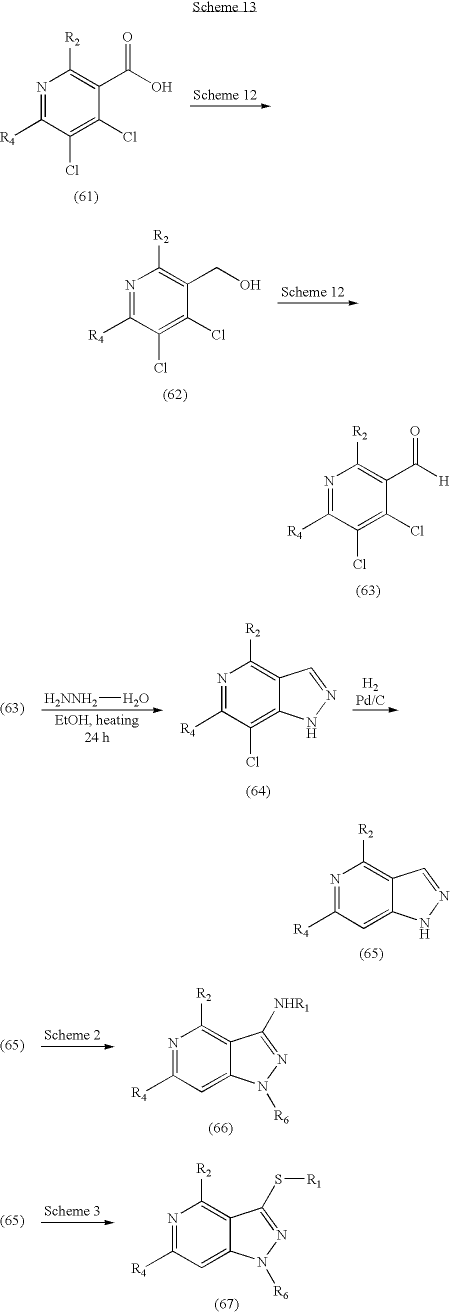

Pyrazolo[4,3-c]pyridines of general formula (66), R₁, R₂, R₄, and R₆ are as defined in formula (I), can be prepared as described in Scheme 13. 3,4-Dichloropyridine-5-carboxylic acids of general formula (61) can be processed as described in Scheme 12 to provide aldehydes of general formula (63). Aldehydes of general formula (63) can be treated with hydrazine hydrate in refluxing ethanol to provide chloropyrazolo[4,3-c]pyridines of general formula (64) as described in Can. J. Chem. (1988) 420. Chloropyrazolo[4,3-c]pyridines of general formula (64) can be treated with a metal transition catalyst such as palladium on carbon under a hydrogen atmosphere to provide pyrazolo[4,3-c]pyridines of general formula (65) as described in JACS (1956) 784. Pyrazolo[4,3-c]pyridines of general formula (65) can be processed as described in Scheme 2 to provide pyrazolo[4,3-c]pyridines of general formula (66).

Pyrazolo[4,3-c]pyridines of general formula (67), R₁, R₂, R₄, and R₆ are as defined in formula (I), can be prepared as described in Scheme 13. Pyrazolo[4,3-c]pyridines of general formula (65) can be processed as described in Scheme 3 to provide pyrazolo[4,3-c]pyridines of general formula (67).

### EXAMPLE 1

### {5-[(1-benzyl-1H-indazol-3-yl)oxy]-2-furyl}methanol

### EXAMPLE A

### 1-benzyl-1H-indazol-3-ol

3-Hydroxyindazole (6.7 g, 50 mmol) and solid sodium hydroxide (8.0 g, 200 mmol) in DMSO (100 mL) were treated with benzyl bromide (6.5 mL, 55 mmol) at room temperature. After stirring for 24 hours, the mixture was treated with water (250 mL) and the resulting solution was acidified with 10% citric acid to pH 4 and extracted with ethyl acetate. The ethyl acetate layer was washed with water, brine, dried with anhydrous MgSO₄, filtered and the filtrate concentrated under reduced pressure to provide 9.5 g of crude product. Diethyl ethyl was added to the crude and the resultant precipitate was filtered. The filtrate was concentrated under reduced pressure and purified by column chromatography (silica gel, 3:1 hexanes:EtOAc) to provide the title compound, 6.7 g (combined yield). ¹H NMR (300 MHz, DMSO-d₆) δ5.36 (s, 2H), 7.00 (t, J=9 Hz, 1H), 7.25 (m, 6H), 7.52 (d, J=9 Hz, 1H), 7.61 (d, J=9 Hz, 1H), 10.70 (s, 1H); MS (APCI+) m/z 225 (M+H)⁺.

### EXAMPLE 1B

### 5-[(1-benzyl-1H-indazol-3-yl)oxy]-2-furaldehyde

The product from Example 1A (120 mg, 0.53 mmol) and 5-nitro-2-furaldehyde (76 mg, 0.54 mmol) in THF (20 mL) were treated with NaH (60% oil dispersion) (22 mg, 0.55 mmol) and the resulting mixture was stirred at ambient temperature for 16 hours. The mixture was concentrated under reduced pressure, acidified with 10% citric acid and extracted with EtOAc. The ethyl acetate layer was washed with water, brine, dried with MgSO₄, filtered and the filtrate concentrated under reduced pressure. The residue was chromatographed (silica gel, 1:1 hexanes:EtOAc) to provide 120 mgs of the title compound. ¹H NMR (300 MHz, DMSO-d₆) δ5.61 (s, 2H), 6.22 (d, J=3 Hz, 1H), 7.00 (t, J=9 Hz, 1H), 7.25 (m, 6H), 7.50 (m, 1H), 7.63 (m, 2H), 7.79 (d, J=9 Hz, 1H), 9.41 (s, 1H); MS (APCI+) m/z 319 (M+H)⁺.

### EXAMPLE 1C

### {5-[(1-benzyl-1H-indazol-3-yl)oxy]-2-furyl}methanol

The product from Example 1B (96 mg, 0.3 mmol) in ethanol (5 mL) was treated with NaBH₄ (8 mg, 0.2 mmol) and refluxed for 30 minutes. The mixture was allowed to cool to room temperature, treated with 10% citric acid and extracted with EtOAc. The ethyl acetate layer was washed with water, brine, dried with MgSO₄, filtered and the filtrate concentrated under reduced pressure. The residue was purified by column chromatography (silica gel, 1:1 hexanes:EtOAc) to provide 90 mg of the title compound. ¹H NMR (300 MHz, DMSO-d₆) δ4.31 (d, J=6 Hz, 2 H), 5.19 (t, J=6 Hz, 1H), 5.55 (s, 2H), 5.84 (d, J=3 Hz, 1H), 6.30 (d, J=3 Hz, 1H), 7.13 (t, J=9 Hz, 1H), 7.25 (m, 5H), 7.45 (m, 2H), 7.70 (d, J=9 Hz, 1H); MS (DCI-NH₃) m/z 321 (M+H)⁺. Analysis calcd for C₁₉H₁₆N₂O₃: C, 71.24; H, 5.03; N, 8.74. Found: C, 71.03; H, 4.97; N, 8.87.

### EXAMPLE 2

### {6-[(1-benzyl-1H-indazol-3-yl)oxy]-2-pyridinyl}methanol

### EXAMPLE 2A

### methyl 6-[(1-benzyl-1H-indazol-3-yl)oxy]-2-pyridinecarboxylate

The product from Example 1A (224 mg, 1 mmol), K₂CO₃ (276 mg, 2 mmol), methyl 6-chloro-2-pyridinecarboxylate (342 mg, 2 mmol) and Cu (25 mg) in pyridine (25 mL) was refluxed for 16 hours. The mixture was allowed to cool to room temperature, concentrated under reduced pressure and the residue was partitioned between water and EtOAc. The ethyl acetate layer was washed with water, brine, dried with MgSO₄, filtered and the filtrate concentrated under reduced pressure. The residue was purified by chromatography (silica gel, 1:1 hexanes:EtOAc) to afford 140 mg of the title compound. mp 92-94° C.; ¹H NMR (300 MHz, DMSO-d₆) δ3.88 (s, 3H), 5.57 (s, 2H), 7.08 (m, 1H), 7.30 (m, 7H), 7.42 (m, 1H), 7.74 (d, J=9 Hz, 1H), 8.26 (dd, J=3 Hz and 9 Hz, 1H), 8.36 (dd, J=3 Hz and 9 HZ, 1H); MS (APCI+) m/z 360 (M+H)⁺. Analysis calcd for C₂₁H₁₇N₃O₃: C, 70.18; H, 4.77; N, 11.69. Found: C, 70.47; H, 4.72; N, 11.59.

### EXAMPLE 2B

### {6-[(1-benzyl-1H-indazol-3-yl)oxy]-2-pyridinyl}methanol

The product from Example 2A (110 mg, 0.3 mmol) and NaBH₄ (38 mg, 1 mmol) in THF (15 mL) was treated with methanol (5 mL) dropwise at 50-55° C. After stirring for 30 minutes, the mixture was concentrated under reduced pressure, dissolved in EtOAc, washed with water, brine and dried with MgSO₄, filtered and the filtrate concentrated. the residue was purified by column chromatography (silica gel, 1:1 hexanes:EtOAc) to provide 70 mg of the title compound. mp 156-158° C.; ¹H NMR (300 MHz, DMSO-d₆) δ4.71 (d, J=7 Hz, 2H), 5.43 (t, J=7 Hz, 1H), 5.57 (s, 2H), 7.05 (t, J=9 Hz, 1H), 7.30 (m, 7H), 7.40 (m, 1H), 7.72 (d, J=9 Hz, 1H), 7.92 (m, 2H); MS (APCI+) m/z 332 (M+H)⁺. Analysis calcd for C₂₀H₁₇N₃O₂: C, 72.49; H, 5.17; N, 12.68. Found: C, 72.15; H, 5.19; N, 12.48.

### EXAMPLE 3

### 5-[(1-benzyl-1H-indazol-3-yl)oxy]-2-furaldehyde (6-chloro-3-pyridazinyl)hydrazone

The product from Example 1B (96 mg, 0.3 mmol) and acetic acid (2 drops) in MeOH (5 mL) and THF (10 mL) were treated with 3-chloro-6-hydrazinopyridazine (46 mg, 0.4 mmol). After stirring at room temperature for 16 hours, the mixture was concentrated under reduced pressure. The residue was dissolved in EtOAc, washed with water, brine, dried with MgSO₄, filtered and the filtrate was concentrated under reduced pressure. The residue was purified by column chromatography (silica gel, 4:1 CH₂Cl₂:EtOAc) to provide 115 mg of the title compound. mp 200-202° C.; ¹H NMR (300 MHz, DMSO-d₆) δ5.57 (s, 2H), 6.10 (d, J=3 Hz, 1H), 6.86 (d, J=3 Hz, 1H), 7.27 (m, 6H), 7.48 (t, J=9 Hz, 2H), 7.60 (dd, J=6 Hz, 9 Hz, 2H), 7.75 (d, J=9 Hz, 1H), 7.92 (s, 1H), 11.63 (s, 1H; MS (APCI+) m/z 445 (M+H)⁺; (APCI−) m/z 443 (M−H)⁻; 479 (M+Cl)⁻; Analysis calcd for C₂₃H₁₇ClN₆O₂.0.75 H₂O: C, 60.26; H, 4.07; N, 18.30. Found: C, 60.30; H, 3.96; N, 17.86.

### EXAMPLE 4

### 5-[(1-benzyl-1H-indazol-3-yl)oxy]-2-furoic acid

### EXAMPLE 4A

### methyl 5-[(1-benzyl-1H-indazol-3-yl)oxy]-2-furoate

The product from Example 1A (224 mg, 1 mmol) and methyl 5-nitro-2-furoate (255 mg, 1.5 mmol) in DMSO (15 mL) were treated with 60% oil dispersion NaH (80 mg, 2 mmol). After refluxing (80° C.) for 4 hours, the mixture was allowed to cool to room temperature, water was added and the mixture was extracted with EtOAc. The ethyl acetate layer was washed with water, brine, dried with MgSO₄, filtered and the filtrate concentrated under reduced pressure. The residue was purified by column chromatography (silica gel, 3:1 hexanes:EtOAc) to provide 300 mg of the title compound. mp 89-91° C.; ¹H NMR (300 MHz, DMSO-d₆) δ3.80 (s, 3H), 5.59 (s, 2H), 6.17 (d, J=4 Hz, 1H), 7.26 (m, 6H), 7.39 (d, J=4 Hz, 1H), 7.48 (m, 1H), 7.61 (d, J=9 Hz, 1H), 7.77 (d, J=9 Hz, 1H); MS (APCI+) m/z 349 (M+H)⁺. Analysis calcd for C₂₀H₁₆N₂O₄: C, 68.96; H, 4.63; N, 8.04. Found: C, 68.24; H, 4.53; N, 7.97.

### EXAMPLE 4B

### 5-[(1-benzyl-1H-indazol-3-yl)oxy]-2-furoic acid

The product from Example 4A (140 mg, 0.4 mmol) in MeOH (15 mL) was treated with 1N NaOH (1 mL, 1 mmol). After stirring at ambient temperature for 2 hours, the mixture was treated with water (10 mL). The methanol was removed under reduced pressure and the aqueous solution was acidified with 10% citric acid to pH 3. The resultant solid was filtered, washed with water and dried under reduced pressure to afford 100 mg of the title compound. mp 162-165° C.; ¹H NMR (300 MHz, DMSO-d₆) δ5.59 (s, 2H), 6.11 (d, J=4 Hz, 1H), 7.26 (m, 7H), 7.48 (m, 1H), 7.60 (d, J=9 Hz, 1H), 7.77 (d, J=9 Hz, 1H), 12.60 (br s, 1H); MS (APCI+) m/z 335 (M+H)⁺.

### EXAMPLE 5

### 5-[(1-benzyl-1H-indazol-3-yl)oxy]-N-[3-(dimethylamino)propyl]-2-furamide

The product from Example 4B (100 mg, ˜0.3 mmol) was treated with 3-dimethylamino-1-propylamine (0.126 mL, 1 mmol), N-hydroxysuccinimide (57 mg, 0.5 mmol) and EDCI (191 mg, 1 mmol) in CHCl₃ (10 mL) and 1,4-dioxane (10 mL) at room temperature. After stirring for 16 hours, the solvents were removed under reduced pressure. The residue was dissolved in EtOAc, washed with water, brine, dried with MgSO₄, filtered and the filtrate concentrated under reduced pressure. The residue was purified by column chromatography (silica gel, 3:1 CH₂Cl₂:EtOH, plus a few drops of concentrated NH₄OH) to provide 55 mg of the title compound. ¹H NMR (300 MHz, DMSO-d₆) δ1.64 (quintet, J=7 Hz, 2 H), 2.20 (s, 6H), 2.37 (t, J=7 Hz, 2 H), 3.22 (m, 2 H), 5.59.(s, 2H), 6.10 (d, J=4 Hz, 1 H), 7.10 (d, J=4 Hz, 1H), 7.26 (m, 6H), 7.48 (m, 1H), 7.55 (d, J=9 Hz, 1H), 7.75 (d, J=9 Hz, 1H), 8.48 (t, J=7 Hz, 1H); MS (APCI+) m/z 419 (M+H)⁺; (APCI−) m/z 453 (M+Cl)⁻.)⁺; Analysis calcd for C₂₄H₂₆N₄O₃ 1.5 H₂O: C, 64.72; H, 6.51; N, 12.58. Found: C, 65.00; H, 6.23; N, 12.59.

### EXAMPLE 6

### 5-[(1-benzyl-1H-indazol-3-yl)oxy]-N-(4-hydroxybutyl)-2-furamide

The product from Example 4B (50 mg, 0.15 mmol) in CHCl₃/1,4-dioxane (1:1) (5 mL) was treated with EDCI (42 mg, 0.22 mmol) and N-hydroxysuccinimide (26 mg, 0.15 mmol). After stirring at ambient temperature for 1 hour, the mixture was treated with 4-amino-1-butanol (133 mg, 0.15 mmol) and the reaction mixture was stirred at ambient temperature for an additional 16 hours. The reaction mixture was diluted with CHCl₃ (5 mL) and the phases separated. The organic layer was washed with water (2 mL), dried over anhydrous Na₂SO₄, filtered and the filtrate concentrated under reduced pressure. The residue was purified by flash chromatograpy (silica gel, 1:1 hexanes:EtOAc) to provide the title compound. ¹H NMR (499.6 MHz, CDCl₃) δ7.60 (d, J=10 Hz, 1H), 7.19 (m, 9H), 6.41 (br s, 1H), 6.00 (d, J=5 Hz, 1H), 5.47 (s, 2H), 3.70 (t, J=5 Hz, 2H), 3.46 (q, J=5 Hz, 2H), 1.67 (m, 4H); MS (ESI) m/z 406 (M+H)⁺.

### EXAMPLE 7

### 5-[(1-benzyl-1H-indazol-3-yl)oxy]-N-[1-(hydroxymethyl)butyl]-2-furamide

The product from Example 4B and 2-amino-1-pentanol were processed according to the procedure described in Example 6 to provide the title compound. ¹H NMR (499.6 MHz, CDCl₃) δ7.62 (d, J=10 Hz, 1H), 7.25 (m, 9H), 6.28 (br d, J=10 Hz, 1H), 6.02 (d, J=5 Hz, 1H), 5.47 (s, 2H), 4.12 (m, 1H), 3.77 (dd, J=3.4, 10 Hz, 1H), 3.68 (dd, J=5, 10 Hz, 1H), 1.59 (m, 2H), 1.43 (m, 2H), 0.95 (t, J=10 Hz, 3H); MS (ESI) m/z 420 (M+H)⁺.

### EXAMPLE 8

### 5-[(1-benzyl-1H-indazol-3-yl)oxy]-N-(5-hydroxy-1,5-dimethylhexyl)-2-furamide

The product from Example 4B and 6-amino-2-methyl-2-heptanol were processed according to the procedure described in Example 6 to provide the title compound. ¹H NMR (499.6 MHz, CDCl₃) δ7.61 (d, J=10 Hz, 1H), 7.24 (m, 9H), 6.01 (d, J=10 Hz, 1H), 5.98 (br d, J=10 Hz, 1H), 5.47 (s, 2H), 4.19 (m, 1H), 1.49 (m, 6H), 1.23 (d, J=10 Hz, 3H), 1.21 (s, 3H), 1.20 (s, 3H); MS (ESI) m/z 462 (M+H)⁺.

### EXAMPLE 9 363987

### 5-[(1-benzyl-1H-indazol-3-yl)oxy]-N-(2-hydroxy-2-phenylethyl)-2-furamide

The product from Example 4B and 2-amino-1-phenylethanol were processed according to the procedure described in Example 6 to provide the title compound. ¹H NMR (499.6 MHz, CDCl₃) δ7.67 (d, J=10 Hz, 1H), 7.59 (d, J=5 Hz, 1H), 7.50 (d, J=5 Hz, 1H), 7.27 (m, 11H), 6.62 (br s, 1H), 6.20 (d, J=5 Hz, 1H), 6.03 (d, J=5 Hz, 1H), 5.47 (d, J=5 Hz, 2H), 4.93 (dd, J=5, 10 Hz, 1H), 3.85 (m, 1H), 3.51 (m, 1H); MS (ESI) m/z 454 (M+H)⁺.

### EXAMPLE 10

### 5-[(1-benzyl-1H-indazol-3-yl)oxy]-N-[2-(4-morpholinyl)ethyl]-2-furamide

The product from Example 4B and 2-amino-1-phenylethanol were processed according to the procedure described in Example 6 to provide the title compound. ¹H NMR (499.6 MHz, CDCl₃) δ7.59 (d, J=5 Hz, 1H), 7.25 (m, 9H), 6.66 (br s, 1H), 5.99 (d, J=5 Hz, 1H), 5.47 (s, 2H), 3.69 (t, J=5 Hz, 4H), 3.51 (q, J=5 Hz, 2H), 2.56 (t, J=5 Hz, 2H), 2.48 (m, 4H); MS (ESI) m/z 447 (M+H)⁺.

### EXAMPLE 11

### 1-benzyl-3-(2-pyridinyloxy)-1H-indazole

The product from Example 1A (100 mg, 0.44 mmol) in DMF (2 mL) was treated with Cs₂CO₃ (286 mg, 0.88 mmol) and 2-fluoropyridine (86 mg, 0.88 mmol) and heated to 110° C. for 16 hours. The reaction mixture was allowed to cool to ambient temperature and diluted with Et₂O (15 mL). The organic layer was washed with water (2 mL), dried over anhydrous Na₂SO₄, filtered and the filtrate concentrated under reduced pressure. The residue was purified by flash chromatography (silica gel, 1:1 hexanes:EtOAc) to provide the title compound. ¹H NMR (499.6 MHz, CDCl₃) δ8.18 (br d, J=3.8 Hz, 1H), 7.72 (m, 1H), 7.44 (d, J=5 Hz, 1H), 7.33 (m, 7H), 7.12 (d, J=5 Hz, 1H), 7.05 (m, 2H), 5.51 (s, 2H); MS (ESI) m/z 302 (M+H)⁺.

### EXAMPLE 12

### 1-benzyl-3-(2-pyrimidinyloxy)-1H-indazole

The product from Example 1A and 2-chloropyrimidine were processed according to the procedure described in Example 11 to provide the title compound. ¹H NMR (499.6 MHz, CDCl₃) δ8.59 (d, J=5 Hz, 2H), 7.48 (d, J=10 Hz, 1H), 7.30 (m, 7H), 7.09-7.12 (m, 2H), 5.55 (s, 2H); MS (ESI) m/z 303 (M+H)⁺.

### EXAMPLE 13

### 1-benzyl-3-{[5-(trifluoromethyl)-3-pyridinyl]oxy}-1H-indazole

The product from Example 1A and 3-chloro-5-trifluoromethylpyridine were processed according to the procedure described in Example 11 to provide the title compound. ¹H NMR (499.6 MHz, CDCl₃) δ7.90 (m, 1H), 7.60 (m, 1H), 7.28 (m, 10H), 5.55 (s, 2H); MS (ESI) m/z 370 (M+H)⁺.

### EXAMPLE 14

### 2-[(1-benzyl-1H-indazol-3-yl)oxy]nicotinamide

The product from Example 1A (100 mg, 0.44 mmol), K₂CO₃ (122 mg, 2 mmol), 2-chloronicotinamide (140 mg, 2 mmol) and Cu (10 mg) in pyridine (10 mL) were combined and refluxed for 16 hours. The mixture was allowed to cool to room temperature and concentrated under reduced pressure. The residue was partitioned between water and EtOAc. The ethyl acetate layer was washed with water, brine, dried with MgSO₄, filtered and the filtrate concentrated under reduced pressure. The residue was purified by flash chromatography (silica gel, 1:1 hexanes:EtOAc) to afford 59 mg of the title compound. ¹H NMR (499.6 MHz, CDCl₃) δ8.65 (dd, J=5, 10 Hz, 1H), 8.20 (m, 1H), 7.76 (br s, 1H), 7.34 (m, 8H), 7.21 dd, J=4.5, 7.5 Hz, 1H), 7.12 (m, 1H), 6.09 (br s, 1H), 5.55 (s, 2H); MS (ESI) m/z 345 (M+H)⁺.

### EXAMPLE 15

### 2-[(1-benzyl-1H-indazol-3-yl)oxy]benzoic acid

The product from Example 1A and 2-bromobenzoic acid were processed according to the procedure described in Example 14 to provide the title compound. The product was isolated by concentrating the pyridine layer and extracting with water (2 mL). The aquoeus layer was then acidified to pH 3.0 with 10% HCl and extracted with EtOAc (5 mL). The EtOAc layer was dried over anhydrous Na₂SO₄, filtered and the filtrate concentrated under reduced pressure to afford 60 mg of the title compound. ¹H NMR (499.6 MHz, CDCl₃) δ8.5 (br s, 1H), 7.78 (s, 1H), 7.57 (d, J=5 Hz, 1H), 7.31 (m, 10H), 7.13 (t, J=5 Hz, 1H), 5.47 (s, 2H); MS (ESI) m/z 345 (M+H)⁺.

### EXAMPLE 16

### 2-[(1-benzyl-1H-indazol-3-yl)oxy]-N-[2-(dimethylamino)ethyl]benzamide

The product from Example 15 and N,N-dimethyl-1,2-ethanediamine were processed according to the procedure described in Example 6 to provide the title compound. ¹H NMR (499.6 MHz, CDCl₃) δ2.85 (s, 6H), 3.15 (m, 2H), 3.58 (m, 2H), 4.54 (m, 1H), 4.76 (m, 1H), 7.30 (m, 11H), 7.79 (m, 2H), 7.97 (br m, 1H); MS (ESI) m/z 415 (M+H)⁺.

### EXAMPLE 17

### N-[3-(4-{2-[(1-benzyl-1H-indazol-3-yl)oxy]benzoyl}-1-piperazinyl)propyl]-N,N-dimethylamine

The product from Example 15 and N,N-dimethyl-N-[3-(1-piperazinyl)propyl]amine were processed according to the procedure described in Example 6 to provide the title compound. ¹H NMR (499.6 MHz, CDCl₃) δ2.35 (m, 4H), 2.83 (s, 6H), 3.20 (m, 5H), 3.47 (m, 3H), 3.79 (m, 1H), 4.00 (m, 1H), 4.55 (d, J=15.0 Hz, 1H), 4.78 (d, J=15.0 Hz, 1H), 4.78 (d, J=15.0 Hz, 1H), 7.24 (m, 6H), 7.41 (m, 2H), 7.50 (m, 3H), 7.83 (d, J=10.0 Hz, 1H); MS (ESI) m/z 498 (M+H)⁺.

### EXAMPLE 18

### 2-[(1-benzyl-1H-indazol-3-yl)oxy]-N-[3-(dimethylamino)propyl]benzamide

The product from Example 15 and N,N-dimethyl-1,3-propanediamine were processed according to the procedure described in Example 6 to provide the title compound. ¹H NMR (499.6 MHz, CDCl₃) δ2.00 (s, 6H), 2.68 (m, 2H), 2.97 (t, J=5.0 Hz, 2H), 3.13 (m, 1H), 3.27 (m, 1H), 4.55 (d, J=15.0 Hz, 1H), 4.95 (d, J=15.0 Hz, 1H), 7.01 (m, 2H), 7.29 (m, 7H), 7.51 (m, 2H), 7.65 (m, 1H), 7.81 (m, 2H); MS (ESI) m/z 429 (M+H)⁺.

### EXAMPLE 19

### 2-[(1-benzyl-1H-indazol-3-yl)oxy]-N-(4-hydroxycyclohexyl)benzamide

The product from Example 15 and 4-aminocyclohexanol were processed according to the procedure described in Example 6 to provide the title compound. ¹H NMR (499.6 MHz, CDCl₃) δ0.85 (m, 1H), 7.88 (m, 1H), 1.32 (m, 4H), 1.72 (m, 1H), 1.95 (m, 2H), 3.52 (m, 1H), 3.67 (m, 1H), 4.48 (d, J=15.0 Hz, 1H), 4.79 (d, J=15.0 Hz, 1H), 7.01 (m, 1H), 7.22 (m, 8H), 7.49 (m, 2H), 7.62 (m, 1H), 7.79 (m, 1H); MS (ESI) m/z 442 (M+H)⁺.

### EXAMPLE 20

### 2-[(1-benzyl-1H-indazol-3-yl)oxy]-N-[(1R,2S)-1-hydroxy-2,3-dihydro-1H-inden-2-yl]benzamide

The product from Example 15 and (1R,2S)-2-amino-2,3-dihydro-1H-inden-1-ol were processed according to the procedure described in Example 6 to provide the title compound. ¹H NMR (499.6 MHz, CDCl₃) δ2.60 (m, 2H), 2.97 (m, 1H), 3.12 (dd, J=5.0, 20.0 Hz, 1H), 4.63 (s, 1H), 4.86 (s, 1H), 5.43 (br s, 1H), 7.48 (m, 17H); MS (ESI) m/z 476 (M+H)⁺.

### EXAMPLE 21

### 2-[(1-benzyl-1H-indazol-3-yl)oxy]-N-[1-(hydroxymethyl)butyl]benzamide

The product from Example 15 and 2-amino-1-pentanol were processed according to the procedure described in Example 6 to provide the title compound. ¹H NMR (499.6 MHz, CDCl₃) δ0.93 (m, 3H), 1.40 (m, 1H), 1.51 (m, 1H), 2.13 (m, 2H), 3.33 (m, 1H), 3.53 (m, 1H), 3.96 (m, 1H), 4.08 (d, J=15.0 Hz, 1H), 4.51 (d, J=15.0 Hz, 1H), 7.50 (m, 13H); MS (ESI) m/z 430 (M+H)⁺.

### EXAMPLE 22

### 2-[(1-benzyl-1H-indazol-3-yl)oxy]-N-(2-hydroxy-2-phenylethyl)benzamide

The product from Example 15 and 2-amino-1-phenylethanol were processed according to the procedure described in Example 6 to provide the title compound. ¹H NMR (499.6 MHz, CDCl₃) δ3.23 (m, 1H), 3.43 (m, 1H), 3.68 (m, 1H), 4.48 (m, 1H), 4.63 (m, 1H), 4.80 (m, 1H), 7.13 (m, 3H), 7.24 (m, 7H), 7.31 (m, 2H), 7.47 (m, 2H), 7.57 (t, J=10.0 Hz, 1H), 7.72 (m, 2H), 7.83 (d, J=5.0 Hz, 1H); MS (ESI) m/z 464 (M+H)⁺.

### EXAMPLE 23

### 2-[(1-benzyl-1H-indazol-3-yl)oxy]-N-[2-(1-methyl-2-pyrrolidinyl)ethyl]benzamide

The product from Example 15 and 2-(1-methyl-2-pyrrolidinyl)ethylamine were processed according to the procedure described in Example 6 to provide the title compound. ¹H NMR (499.6 MHz, CDCl₃) δ1.86 (s, 3H), 2.04 (m, 5H), 2.51 (m, 1H), 2.71 (m, 2H), 3.19 (m, 1H), 3.31 (m, 1H), 3.84 (m, 1H), 4.58 (d, J=15.0 Hz, 1H), 5.04 (d, J=15.0 Hz, 1H), 6.92 (m, 1H), 7.05 (m, 1H), 7.28 (m, 5H), 7.50 (m, 3H), 7.72 (m, 1H), 7.79 (m, 1H), 7.87 (m, 1H); MS (ESI) m/z 455 (M+H)⁺.

### EXAMPLE 24

### 2-[(1-benzyl-1H-indazol-3-yl)oxy]-N-[3-(1H-imidazol-1-yl)propyl]benzamide

The product from Example 15 and 3-(1H-imidazol-1-yl)propylamine were processed according to the procedure described in Example 6 to provide the title compound. ¹H NMR (499.6 MHz, CDCl₃) δ1.74 (m, 1H), 1.82 (m, 1H), 3.10 (m, 1H), 3.19 (m, 1H), 3.94 (m, 2H), 4.53 (d, J=15.0 Hz, 1H), 4.98 (d, J=15.0 Hz, 1H), 6.98 (m, 2H), 7.25 (m, 7H), 7.55 (m, 2H), 7.68 (m, 2H), 7.82 (m, 2H), 8.55 (s, 1H); MS (ESI) m/z 452 (M+H)⁺.

### EXAMPLE 25

### 2-[(1-benzyl-1H-indazol-3-yl)oxy]-N′-(4-morpholinyl)benzohydrazide

The product from Example 15 and 4-morpholinamine were processed according to the procedure described in Example 6 to provide the title compound. ¹H NMR (499.6 MHz, CDCl₃) δ2.48 (m, 2H), 2.63 (m, 2H), 3.69 (m, 4H), 4.47 (d, J=15.0 Hz, 1H), 4.81 (d, J=15.0 Hz, 1H), 7.04 (m, 2H), 7.24 (m, 6H), 7.50 (m, 2H), 7.61 (m, 1H), 7.81 (m, 1H), 7.89 (d, J=5.0 Hz, 1H); MS (ESI) m/z 429 (M+H)⁺.

### EXAMPLE 26

### 2-[(1-benzyl-1H-indazol-3-yl)oxy]-N-[(2-hydroxycyclohexyl)methyl]benzamide

The product from Example 15 and 2-(aminomethyl)cyclohexanol were processed according to the procedure described in Example 6 to provide the title compound. ¹H NMR (499.6 MHz, CDCl₃) δ1.30 (m, 5H), 1.74 (m, 1H), 2.56 (m, 1H), 2.88 (m, 1H), 3.21 (m, 1H), 3.80 (m, 1H), 3.97 (m, 1H), 4.48 (d, J=15.0 Hz, 2H), 4.73 (d, J=15.0 Hz, 1H), 4.82 (d, J=15.0 Hz, 1H), 6.90 (m, 1H), 7.05 (m, 1H), 7.38 (m, 6H), 7.71 (m, 1H), 7.87 (d, J=10.0 Hz, 2H), 8.11 (m, 1H), 8.03 (m, 1H), 7.87 (d, J=10.0 Hz, 2H); MS (ESI) m/z 456 (M+H)⁺.

### EXAMPLE 27

### 3-(benzyloxy)-1-[3-phenyl-2-propenyl]-1H-indazole

The product from Example 1A (70 mg, 0.31 mmol), cinnamyl bromide (99 mg, 0.5 mmol) and KOH (28 mg, 0.5 mmol) in DMSO (10 mL) were stirred at room temperature for 20 hours. The mixture was poured in water and extracted with ethyl acetate. The acetate layer was washed with water, 10% citric acid, brine, dried with anhydrous MgSO₄, filtered and the filtrate concentrated under reduced pressure. The residue was purified by column chromatography (silica gel, 17:3 hexanes:Et₂O) to provide 75 mg (71%) of the title compound. ¹H NMR (300 MHz, DMSO-d₆) δ5.05 (d, J=6 Hz, 2H), 5.42 (s, 2H), 6.40 (m, 1H), 6.55 (m, 1H), 7.06 (m, 1H), 7.33 (m, 9H), 7.60 (m, 4H); MS (APCI+) m/z 341 (M+H)⁺.

### EXAMPLE 28

### 5-{[3-(benzyloxy)-1H-indazol-1-yl]methyl}-2-furoic acid

### EXAMPLE 28A

### ethyl 5-{[3-(benzyloxy)-1H-indazol-1-yl]methyl}-2-furoate

3-Hydroxyindazole (336 mg, 1.5 mmol), ethyl 5-(chloromethyl)-2-furoate (0.32 mL, 2 mmol) and solid NaOH (80 mg, 2 mmol) in anhydrous DMSO (15 mL) were stirred at room temperature for 14 hours. The mixture was poured into icy water and extracted with EtOAc. The extract was washed with water, brine, dried with anhydrous MgSO₄, filtered and the filtrate concentrated under reduced pressure. The residue was purified by chromatography (silica gel, 3:1 hexanes:EtOAc) to afford 450 mg (79%) of title compound. ¹H NMR (300 MHz, DMSO-d₆) δ1.24 (t, J=7 Hz, 3H), 4.23 (q, J=7 Hz, 2H), 5.37 (s, 2H), 5.56 (s, 2H), 6.48 (d, J=3 Hz, 1H), 7.09 (m, 1H), 7.20 (d, J=3 Hz, 1H), 7.38 (m, 4H), 7.50 (m, 2H), 7.62 (m, 2H); MS (APCI+) m/z 377 (M+H)⁺. Analysis calcd for C₂₂H₂₀N₂O₄: C, 70.20; H, 5.36; N, 7.44. Found: C, 69.89; H, 5.27; N, 7.25.

### EXAMPLE 28B

### 5-{[3-(benzyloxy)-1H-indazol-1-yl]methyl}-2-furoic acid

The product from Example 28A (40 mg, 0.1 mmol) in EtOH (5 mL) was treated with 1N NaOH (0.25 mL, 0.25 mmol) at room temperature. After stirring for 2 hours, the mixture was treated with water (10 mL), acidified with 10% citric acid to pH 3, extracted with EtOAc and concentrated under reduced pressure to provide 35 mg (˜99%) of title compound. ¹H NMR (300 MHz, DMSO-d₆) δ5.37 (s, 2H), 5.56 (s, 2H), 6.48 (d, J=3 Hz, 1H), 7.08 (m, 1H), 7.12 (d, J=3 Hz, 1H), 7.38 (m, 4H), 7.51 (m, 2H), 7.62 (m, 2H), 13.05 (br s, 1H); MS (APCI+) m/z 349 (M+H)⁺; Analysis calcd for C₂₂H₁₆N₂O₄: C, 68.96; H, 4.63; N, 8.04. Found: C, 68.69; H, 4.64; N, 7.93.

### EXAMPLE 29

### (5-{[3-(benzyloxy)-1H-indazol-1-yl]methyl}-2-furyl)methanol

The product from Example 28A (113 mg, 0.3 mmol) and NaBH₄ (38 mg, 1 mmol) in THF (15 mL) were treated with MeOH dropwise at 50° C. After stirring at 50° C. for 15 minutes, the mixture was allowed to cool to room temperature and then treated with 10% Citric acid and extracted with EtOAc. The extract was washed with water, brine, dried with anhydrous MgSO₄, filtered and the filtrate concentrated under reduced pressure. The residue was purified by chromatography (silica gel, 1:1 hexanes:EtOAc) to afford 90 mg (89%) of the title compound. mp 80-82° C.; ¹H NMR (300 MHz, DMSO-d₆) δ4.30 (d, J=6 Hz, 2H), 5.14 (t, J=6 Hz, 1H), 5.38 (s, 2H), 5.43(s, 2H), 6.20 (d, J=3 Hz, 1H), 6.30 (d, J=3 Hz, 1H), 7.05 (t, J=8 Hz, 1H), 7.38 (m, 4H), 7.52 (m, 2H), 7.60 (m, 2H); MS (APCI+) m/z 335 (M+H)⁺; Analysis calcd for C₂₀H₁₈N₂O₃: C, 71.84; H, 5.43; N, 8.38. Found: C, 71.74; H, 5.42; N, 8.32.

### EXAMPLE 30

### 3-(benzyloxy)-1-({5-[(4-methyl-1-piperazinyl)carbonyl]-2-furyl}methyl)-1H-indazole

The product from Example 28B (348 mg, 1 mmol), HOBt (135 mg. 1 mmol), 1-methylpiperazine (150 mg, 1.5 mmol) and EDCI (290 mg, 1.5 mmol) in CH₂Cl₂ (30 mL) were stirred at room temperature for 12 hours. The mixture was washed with water, brine, dried with MgSO₄, filtered and the filtrate concentrated under reduced pressure. The residue was purified by column chromatography (silica gel, EtOAc:MeOH 4:1, plus 1% Et₃N) to provide 350 mg (80%) of the title compound. ¹H NMR (300 MHz, DMSO-D₆) δ2.12 (s, 3H), 2.18 (s, 4H), 3.45 (t, 4H), 5.37 (s, 2H), 5.55 (s, 2H), 6.5 (d, J=3 Hz, 1H), 6.9 (d, J=3 Hz, 1H), 7.09 (t, J=7.5 Hz, 1H), 7.38 (m, 4H), 7.5 (dd, J=6 Hz, 2H), 7.55 (dd, J=9 Hz, 2H); MS (DCI-NH₃) m/z 431.2 (M+H)⁺; FAB-Exact mass calcd for C₂₅H₂₇N₄O₃ (M+H)⁺ 431.2083; Found 431.2080.

### EXAMPLE 31

### [1-(5-{[3-(benzyloxy)-1H-indazol-1-yl]methyl}-2-furoyl)-2-pyrrolidinyl]methanol

The product from Example 28B and 2-pyrrolidinylmethanol were processed according to the procedure described in Example 30 to provide the title compound. ¹H NMR (300 MHz, DMSO-D₆) δ1.8 (m, 4H), 3.44 (m, 3H), 4.05 (m, 1H), 4.72 (t, J=6 Hz, 1H), 5.37 (s, 2H), 5.55 (s, 2H), 6.5 (d, J=3 Hz, 1H), 6.9 (d, J=3 Hz, 1H), 7.09 (t, J=7.5 Hz, 1H), 7.38 (m, 4H), 7.5 (dd, J=6 Hz, 2H), 7.55 (dd, J=9 Hz, 2H); MS (DCI-NH₃) m/z 432(M+H)⁺.

### EXAMPLE 32

### 5-{[3-(benzyloxy)-1H-indazol-1-yl]methyl}-N-[3-(dimethylamino)propyl]-2-furamide

The product from Example 28B and N,N-dimethyl-1,3-propanediamine were processed according to the procedure described in Example 30 to provide the title compound. ¹H NMR (300 MHz, DMSO-D₆) δ1.59 (m, 2H), 2.1 (s, 6H), 2.22 (t, J=6 Hz, 2H), 3.20 (m, 2H), 5.37 (s, 2H), 5.55 (s, 2H), 6.5 (d, J=3 Hz, 1H), 6.9 (d, J=3 Hz, 1H), 7.09 (t, J=7.5 Hz, 1H), 7.38 (m, 4H), 7.5 (dd, J=6 Hz, 2H), 7.55 (dd, J=9 Hz, 2H), MS (DCI-NH₃) m/z 433 (M+H)⁺.

### EXAMPLE 33

### 5-{[3-(benzyloxy)-1H-indazol-1-yl]methyl}-N-(4-hydroxycyclohexyl)-2-furamide

The product from Example 28B and 4-aminocyclohexanol were processed according to the procedure described in Example 30 to provide the title compound. ¹H NMR (300 MHz, DMSO-D₆) δ1.25 (m, 4H), 1.79 (m, 4H), 3.35 (m, 2H), 3.61 (m, 1H), 4.53 (d, J=4.5 Hz, 1H), 5.39 (s, 2H), 5.55 (s, 2H), 6.35 (d, J=3 Hz, 1H), 7.02 (d, J=3 Hz, 1H), 7.09 (t, J=7.5 Hz, 1H), 7.38 (m, 4H), 7.5 (dd, J=6 Hz, 2H), 7.65 (dd, J=9 Hz, 2H), 7.90 (d, J=9 Hz, 1H); MS (DCI-NH₃) m/z 446 (M+H)⁺.

### EXAMPLE 34

### 1-(2-fluorobenzyl)-3-[(5-nitro-2-pyridinyl)oxy]-1H-pyrazolo[3,4-b]pyridine

### EXAMPLE 34A

### methyl 2-chloronicotinate

2-Chloronicotinic acid (4.73 g, 30 mmol) was dissolved in methanol (100 mL) and treated with concentrated H₂SO₄ (2.5 mL). The mixture was refluxed for 4 hours, allowed to cool to room temperature, and concentrated under reduced pressure. The residue was partitioned between a saturated solution of NaHCO₃ and ethyl acetate. The ethyl acetate layer was separated, washed with water, brine, dried over anhydrous MgSO₄, filtered, and the filtrate was concentrated under reduced pressure to provide the title compound. MS (DCI/NH₃) m/z 172 (M+H)⁺.

### EXAMPLE 34B

### 1H-pyrazolo[3,4-b]pyridin-3-ol

Methyl 2-chloronicotinate (5.1 g, ˜30 mmol), Cu (1 g), and hydrazine hydrate were combined in pyridine (100 mL) and refluxed for 10 hours. The mixture was allowed to cool to room temperature and concentrated under reduced pressure. The residue was filtered through a short silica gel column (CH₂Cl₂:MeOH 3:1) to provide the title compound. MS (DCI/NH₃) m/z 136 (M+H)⁺.

### EXAMPLE 34C

### 1-(2-fluorobenzyl)-3-[(2-fluorobenzyl)oxy]-1H-pyrazolo[3,4-b]pyridine

1H-Pyrazolo[3,4-b]pyridin-3-ol (4 g, 29 mmol) in DMSO (25 mL) was treated with solid NaOH (2 g, 50 mmol) and 2-fluorobenzyl chloride (6.3 mL, 35 mmol) at room temperature for 18 hours. The mixture was then poured into H₂O and extracted with ethyl acetate. The acetate layer was washed with water, brine, dried over anhydrous MgSO₄, filtered, and the filtrate was concentrated under reduced pressure. The residue was purified by column chromatography (EtOAc as eluent) to provide the title compound. mp 57-58° C. ¹H NMR (300 MHz, DMSO-d₆) δ4.43 (s, 2H), 5.07 (s, 2H), 7.17 (m, 6H), 7.33 (m, 1H), 7.45 (m, 1H), 7.60 (m, 1H), 8.17 (d-d, J=6 Hz and 3 Hz, 1H), 8.55 (d-d, J=6 Hz and 3 Hz, 1H); MS (DCI/NH₃) m/z 352 (M+H)⁺. Anal. calcd for C₂₀H₁₅F₂N₃O: C, 68.37; H, 4.30; N, 11.96. Found: C, 68.26; H, 4.28; N, 11.73.

### EXAMPLE 34D

### 1-(2-fluorobenzyl)-1H-pyrazolo[3,4-b]pyridin-3-ol

1-(2-Fluorobenzyl)-3-[(2-fluorobenzyl)oxy]-1H-pyrazolo[3,4-b]pyridine (2.46 g, 7 mmol) was suspended in acetic acid (5 mL) and treated with 32% HBr in acetic acid (10 mL). The mixture was stirred at room temperature for 2 hours, poured into icy water (25 mL), and treated with solid NaHCO₃ to pH 5. The mixture was filtered and the filter cake was washed with cold water and dried under reduced pressure to provide the title compound. ¹H NMR (300 MHz, DMSO-d₆) δ5.47 (s, 2H), 7.10 (m, 3H), 7.20 (m, 1H), 7.33 (m, 1H), 8.11(d-d, J=6 Hz and 3 Hz, 1H), 8.47 (d-d, J=6 Hz and 3 Hz, 1H), 11.35 (broad s, 1H); MS (DCI/NH₃) m/z 244 (M+H)⁺.

### EXAMPLE 34E

### 1-(2-fluorobenzyl)-3-[(5-nitro-2-pyridinyl)oxy]-1H-pyrazolo[3,4-b]pyridine

1-(2-Fluorobenzyl)-1H-pyrazolo[3,4-b]pyridin-3-ol (243 mg, 1 mmol), 2-chloro-5-nitropyridine (238 mg, 1.5 mmol), anhydrous K₂CO₃, CuI (10 mg), and Cu (10 mg) were combined in DMF (10 mL) and refluxed for 10 hours. The mixture was poured into water and extracted with ethyl acetate. The acetate layer was washed with water, brine, dried over anhydrous MgSO₄, filtered, and the filtrate was concentrated under reduced pressure. The residue was purified by chromatography (hexanes:ethyl acetate 1:1) to provide the title compound. mp 111-113° C. ¹H NMR (300 MHz, DMSO-d₆) δ5.70 (s, 2H), 7.20 (m, 4H), 7.35 (m, 1H), 7.50 (d, J=9 Hz, 1H), 8.00 (d-d, J=9 Hz and 2 Hz, 1H), 8.66 (d-d, J=6 Hz and 2 Hz, 1H), 8.73 (d-d, J=9 Hz and 3 Hz, 1H), 9.02 (d, J=3 Hz, 1H); MS (DCI/NH₃) m/z 366 (M+H)⁺. Anal. calcd for C₁₈H₁₂FN₅O₃: C, 59.18; H, 3.31; N, 19.17. Found: C, 58.21; H, 3.15; N, 19.33.

### EXAMPLE 35

### 1-(2-fluorobenzyl)-3-[(3-nitro-2-pyridinyl)oxy]-1H-pyrazolo[3,4-b]pyridine

The product from Example 34D and 2-chloro-3-nitropyridine were processed according to the procedure described in Example 34E to provide the title compound. mp 115-117° C. ¹H NMR (300 MHz, DMSO-d₆) δ5.70 (s, 2H), 7.20 (m, 4H), 7.37 (m, 1H), 7.47 (d-d, J=9 Hz and 4 Hz, 1H), 8.03 (d-d, J=9 Hz and 2 Hz, 1H), 8.40 (d-d, J=6 Hz and 2 Hz, 1H), 8.66 (m, 2H); MS (DCI/NH₃) m/z 366 (M+H)⁺. Anal. calcd for C₁₈H₁₂FN₅O₃.0.3H₂O: C, 58.32; H, 3.43; N, 18.89. Found: C, 58.19; H, 3.34; N, 19.14.

### Guanylate Cyclase Assay

Sf9 cells were cultured in Sf-90011 medium supplemented with 10% (v/v) fetal calf serum at 28° C. to give a count of 6×10⁵ cells/ml. The cells were then infected with a mixture of the virus stocks for the α1 and β1 subunits of guanylate cyclase at a multiplicity of infection of 0.1 pfu/cell. The cells were harvested 72 hours after infection and were pelleted by centrifugation. The resulting cell pellets were homogenized with Polytron homogenizer in 3 volumes of 20 mM Tris-HCl (pH 7.6) containing 1 mM EDTA, 5 mM dithiothreitol, 90 mM NaCl, 10% glycerol, and protease inhibitors mix (Roche Molecular Biochemicals). The homogenate was centrifuged at 50,000×g for 60 minutes and the supernatant was aliquotted and stored at −80° C.

Compounds of the present invention were dissolved in DMSO to make 100 mM and saved as stocks at −20° C. When compounds were tested, 10-fold concentrated solutions with 10% DMSO were prepared. 10 μl of 10-fold concentrated compound solutions were added to the reaction mixture total of 100 μl.

Guanylate cyclase activity was measured by the formation of cyclic GMP from GTP in a total volume of 100 μl. Guanylate cyclase-expressed Sf9 cell high-speed supernatant (Sf9 high-speed sup) was used as an enzyme source for the assay. The reaction mixture containing 0.1 μl enzyme of Sf9 high-speed sup, compound solution, 50 mM Tris-HCl (pH 7.6), 4 mM MgCl₂, 0.5 mM IBMX, 15 mM creatine phosphate, 20 μg creatine phosphokinase, 0.1% BSA and 1 mM GTP were incubated at 37° C. for 15 minutes. After the reaction, 0.9 ml of 50 mM sodium acetate buffer (pH 5.8) was added followed by boiling for 3 minutes to stop the reaction. The solution was centrifuged at 3000 rpm for 15 minutes at room temperature and cyclic GMP in 20 μl of the resulting supernatant was measured according to the protocol of Amersham cyclic GMP enzymeimmunoassay system (Amersham, RPN 226).

Compounds of the present invention were tested at 100 μM with and without 1 μM of sodium nitroprusside (SNP). YC-1 was included as a standard for each assay. The data was normalized with the activity of 100 μM YC-1 (without SNP) as 100%. Mean basal efficacy, in Table 1, was expressed as a percentage of 100 μM YC-1 (without SNP).

The mean activation, in Table 1, was calculated based on the formula: (activity of compound of the present invention at 100 μM with 1 μM SNP)−(activity of compound of on at 100 μM)/(activity of SNP at 1 μM)−(basal activity of sGC).

TABLE 1

| | Mean basal | Mean efficacy at | |
|---|---|---|---|
| | efficacy at 100 | 100 μM with | |
| Example | μM, | 1 μM of SNP, | Mean activation at |
| Number | [% of YC-1] | [% of YC-1] | 100 μM |
| YC-1 | 100 | 224 | 2.7 |
| 1 | 9 | 38 | 2.2 |
| 2 | 0 | 20 | 1.8 |
| 3 | 0 | 17 | 1.7 |
| 4 | 0 | 26 | 1.5 |
| 5 | 4 | 65 | 1.7 |
| 6 | 3 | 18 | 2.5 |
| 7 | 3 | 25 | 3.1 |
| 8 | 0 | 9 | 1.5 |
| 9 | 0 | 13 | 1.6 |
| 10 | 1 | 11 | 2.0 |
| 11 | 9 | 70 | 2.8 |
| 12 | −1 | 43 | 2.6 |
| 13 | 3 | 15 | 1.4 |
| 14 | 0 | 23 | 1.8 |
| 15 | 4 | 47 | 2.6 |
| 16 | 6 | 15 | 1.9 |
| 17 | 4 | 14 | 2.0 |
| 18 | 5 | 10 | 1.5 |
| 19 | 3 | 12 | 1.9 |
| 20 | 6 | 11 | 1.5 |
| 21 | 5 | 15 | 1.9 |
| 22 | 2 | 13 | 2.1 |
| 23 | 3 | 15 | 2.2 |
| 24 | 5 | 12 | 1.7 |
| 25 | 4 | 14 | 1.9 |
| 26 | 1 | 12 | 2.0 |
| 27 | −2 | 18 | 1.7 |
| 28 | 6 | 27 | 1.9 |
| 29 | 1 | 11 | 1.4 |
| 34 | 4 | 7 | 1.0 |
| 35 | 7.1 | 28.1 | 1.9 |

The data in Table 1 indicates that compounds of the present invention potentiate the activation of sGC by nitric oxide resulting in increased levels of cGMP. Activation of sGC by allosteric activators or non NO-donors potentiates the effect of NO released during sexual stimulation increasing cGMP. Therefore, compounds of the present invention are useful for sexual dysfunction in mammals including male erectile dysfunction.

Compounds of the present invention can be used in combination with phosphodiesterase 5 inhibitors including, but not limited to, sildenafil or vardenafil as a method of treating sexual dysfunction in a mammal.

Compounds of the present invention can be used in combination with an adrenergic receptor antagonist including, but not limited to, terazosin, prazosin or tamsulosin as a method of treating sexual dysfunction in a mammal.

Compounds of the present invention can be used in combination with a dopamine agonist including, but not limited to, apomorphine as a method of treating sexual dysfunction in a mammal.

Compounds of the present invention can be useful for disorders associated with low levels of cGMP such as cardiovascular disease, atherosclerosis, angina pectoris, diastolic dysfunction, benign prostatic hyperplasia (BPH), incontinence, sexual dysfunction, endothelial dysfunction, trombosis, diabetes, liver cirhosis, cognitive disorders, Alzheimer's disease, anxiety, stress, depression, sleep disorders, migraine, cerebral ischemia, brain trauma, pain, memory and learning disorders as discussed in S. Moncada, E. A. Higgs, FASEB J., 9(13), 1319-1330 (1995); I. S. Severina, Biochemistry (Mosc), 63(7), 794-801 (1998); Y-C. Lee, E. Martin, F. Murad, PNAS, 97(20), 10763-10768 (2000); A. J. Hobbs, TIPS, 18, 484-491 (1997); F. Murad, Adv. Pharmacol., 26, 1-335 (1994); and J. W. Denninger, M. A. Marletta, Biochim. Biophys. Acta, 1411, 334-350 (1999).

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The present invention provides pharmaceutical compositions which comprise compounds of the present invention formulated together with one or more non-toxic pharmaceutically acceptable carriers. The pharmaceutical compositions can be formulated for oral administration in solid or liquid form, for parenteral injection or for rectal administration.

Dosage forms for topical administration of a compound of the present invention include powders, sprays, ointments and inhalants. The active compound is mixed under sterile conditions with a pharmaceutically acceptable carrier and any needed preservatives, buffers or propellants which can be required. Opthalmic formulations, eye ointments, powders and solutions are also contemplated as being within the scope of this invention.

Actual dosage levels of active ingredients in the pharmaceutical compositions of this invention can be varied so as to obtain an amount of the active compound(s) which is effective to achieve the desired therapeutic response for a particular patient, compositions, and mode of administration. The selected dosage level will depend upon the activity of the particular compound, the route of administration, the severity of the condition being treated, and the condition and prior medical history of the patient being treated.

When used in the above or other treatments, a therapeutically effective amount of one of the compounds of the present invention can be employed in pure form or, where such forms exist, in pharmaceutically acceptable salt, ester, amide, or prodrug form. Alternatively, the compound can be administered as a pharmaceutical composition containing the compound of interest in combination with one or more pharmaceutically acceptable carriers. The phrase “therapeutically effective amount” of the compound of the present invention means a sufficient amount of the compound to treat disorders, at a reasonable benefit/risk ratio applicable to any medical treatment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The total daily dose of the compounds of the present invention administered to a mammal, and particularly a human, may range from about 0.003 to about 100 mg/kg/day. For purposes of oral administration, more preferable doses can be in the range of from about 0.01 to about 10 mg/kg/day. If desired, the effective daily dose can be divided into multiple doses for purposes of administration; consequently, single dose compositions may contain such amounts or submultiples thereof to make up the daily dose.

The present invention also provides pharmaceutical compositions that comprise compounds of the present invention formulated together with one or more non-toxic pharmaceutically acceptable carriers. The pharmaceutical compositions can be specially formulated for oral administration in solid or liquid form, for parenteral injection or for rectal administration.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration which include intravenous, intramuscular, intraperitoneal, intrasternal, subcutaneous and intraarticular injection and infusion.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form can be brought about by the inclusion of agents which delay absorption such as aluminum monostearate and gelatin.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which can be prepared by mixing the compounds of the present invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

The present invention contemplates pharmaceutically active compounds either chemically synthesized or formed by in vivo biotransformation of a prodrug, ester, or amide to compounds of formula I.

The compounds of the invention can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

The term “pharmaceutically acceptable salt, ester, amide, and prodrug,” as used herein, refers to carboxylate salts, amino acid addition salts, zwitterions, esters, amides, and prodrugs of compounds of formula I which are within the scope of sound medical judgement, suitable for use in contact with the tissues of humans and lower animals without undue toxicity, irritation, allergic response, and the like, are commensurate with a reasonable benefit/risk ratio, and are effective for their intended use.

The compounds of the present invention can be used in the form of pharmaceutically acceptable salts derived from inorganic or organic acids. The term “pharmaceutically acceptable salt” means those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. The salts can be prepared in situ during the final isolation and purification of the compounds of the present invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as maleic acid, fumaric acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Preferred salts of the compounds of the present, invention include phosphate, tris and acetate.

The term “pharmaceutically acceptable prodrug” or “prodrug,” as used herein, represents those prodrugs of the compounds of the present invention which are, within the scope of sound medical judgement, suitable for use in contact with the tissues of humans and lower animals without undue toxicity, irritation, allergic response, and the like, commensurate with a reasonable benefit/risk ratio, and effective for their intended use. Prodrugs of the present invention may be rapidly transformed in vivo to compounds of formula I, for example, by hydrolysis in blood.

The term “pharmaceutically acceptable ester” or “ester,” as used herein, refers to esters of compounds of the present invention which hydrolyze in vivo and include those that break down readily in the human body to leave the parent compound or a salt thereof. Examples of pharmaceutically acceptable, non-toxic esters of the present invention include C₁-to-C₆ alkyl esters and C₅-to-C₇ cycloalkyl esters, although C₁-to-C₄ alkyl esters are preferred. Esters of the compounds of formula I may be prepared according to conventional methods.

The term “pharmaceutically acceptable amide” or “amide,” as used herein, refers to non-toxic amides of the present invention derived from ammonia, primary C₁-to-C₆ alkyl amines and secondary C₁-to-C₆ dialkyl amines. In the case of secondary amines, the amine may also be in the form of a 5- or 6-membered heterocycle containing one nitrogen atom. Amides derived from ammonia, C₁-to-C₃ alkyl primary amides and C₁-to-C₂ dialkyl secondary amides are preferred. Amides of the compounds of formula I may be prepared according to conventional methods.

What is claimed is:

1. A compound of formula (I) 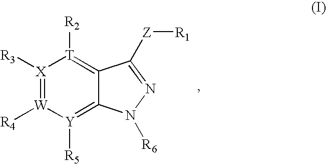
or a pharmaceutically acceptable salt, ester, amide or prodrug thereof, wherein
T, X, W, and Y are independently selected from the group consisting of C and N provided that at most, only two of T, X, W, and Y can be nitrogen at the same time;
Z is selected from the group consisting of O, S and N(R₇);
R₁ is selected from the group consisting of aryl, arylalkenyl, arylalkyl, heterocycle, heterocyclealkenyl and heterocyclealkyl;
R₂ and R₄ are independently selected from the group consisting of hydrogen, alkenyl, alkoxy, alkoxycarbonyl, alkyl, alkylcarbonyl, alkylcarbonyloxy, alkylthio, alkynyl, carboxy, cyano, formyl, halogen, haloalkoxy, haloalkyl, hydroxy, hydroxyalkyl, mercapto, nitro, —NZ₁Z₂, (NZ₁Z₂)carbonyl and (NZ₁Z₂)sulfonyl wherein Z₁ and Z₂ are independently selected from the group consisting of hydrogen, alkyl, alkylcarbonyl and formyl;
R₃ and R₅ are independently absent or selected from the group consisting of hydrogen, alkenyl, alkoxy, alkoxycarbonyl, alkyl, alkylcarbonyl, alkylcarbonyloxy, alkylthio, alkynyl, carboxy, cyano, formyl, halogen, haloalkoxy, haloalkyl, hydroxy, hydroxyalkyl, mercapto, nitro, —NZ₁Z₂, (NZ₁Z₂)carbonyl and (NZ₁Z₂)sulfonyl wherein Z₁ and Z₂ are independently selected from the group consisting of hydrogen, alkyl, alkylcarbonyl and formyl;
R₆ is selected from the group consisting of aryl, arylalkenyl, arylalkyl, heterocycle, heterocyclealkenyl and heterocyclealkyl; and
R₇ is selected from the group consisting of hydrogen and alkyl.

2. A compound according to claim 1 wherein
T, X, W, and Y are C;
Z is O;
R₁ is selected from the group consisting of aryl, arylalkyl and heterocycle; and
R₆ is selected from the group consisting of arylalkenyl, arylalkyl, heterocycle and heterocyclealkyl.

3. A compound according to claim 1 wherein
T, X, W, and Y are C;
Z is O;
R₁ is heterocycle; and
R₆ is arylalkyl.

4. A compound according to claim 1 wherein
T, X, W, and Y are C;
Z is O;
R₁ is heterocycle selected from the group consisting of furyl, pyridinyl and pyrimidinyl wherein the heterocycle is substituted with 0, 1 or 2 substituents selected from the group consisting of carboxy, haloalkyl, hydroxyalkyl and (NR_{A}R_{B})carbonyl wherein R_{A} and R_{B} are independently selected from the group consisting of hydrogen, arylhydroxyalkyl, heterocyclealkyl, hydroxyalkyl and (NZ₁Z₂)alkyl;
R₂, R₃, R₄ and R₅ are hydrogen; and
R₆ is phenylmethyl.

5. A compound according to claim 4 selected from the group consisting of
{5-[(1-benzyl-1H-indazol-3-yl)oxy]-2-furyl}methanol;
{6-[(1-benzyl-1H-indazol-3-yl)oxy]-2-pyridinyl}methanol;
5-[(1-benzyl-1H-indazol-3-yl)oxy]-2-furoic acid;
5-[(1-benzyl-1H-indazol-3-yl)oxy]-N-[3-(dimethylamino)propyl]-2-furamide;
5-[(1-benzyl-1H-indazol-3-yl)oxy]-N-(4-hydroxybutyl)-2-furamide;
5-[(1-benzyl-1H-indazol-3-yl)oxy]-N-[1-(hydroxymethyl)butyl]-2-furamide;
5-[(1-benzyl-1H-indazol-3-yl)oxy]-N-(5-hydroxy-1,5-dimethylhexyl)-2-furamide;
5-[(1-benzyl-1H-indazol-3-yl)oxy]-N-(2-hydroxy-2-phenylethyl)-2-furamide;
5-[(1-benzyl-1H-indazol-3-yl)oxy]-N-[2-(4-morpholinyl)ethyl]-2-furamide;
1-benzyl-3-(2-pyridinyloxy)-1H-indazole;
1-benzyl-3-(2-pyrimidinyloxy)-1H-indazole;
1-benzyl-3-{[5-(trifluoromethyl)-3-pyridinyl]oxy}-1H-indazole; and
2-[(1-benzyl-1H-indazol-3-yl)oxy]nicotinamide.

6. A compound according to claim 1 wherein
T, X, W, and Y are C;
Z is O;
R₁ is aryl; and
R₆ is arylalkyl.

7. A compound according to claim 1 wherein
T, X, W, and Y are C;
Z is O;
R₁ is phenyl substituted with 0, 1, or 2 substituents selected from the group consisting of carboxy, heterocyclecarbonyl and (NR_{A}R_{B})carbonyl wherein R_{A} and R_{B} are independently selected from the group consisting of hydrogen, aryl, arylhydroxyalkyl, cycloalkyl, cycloalkylalkyl, heterocycle, heterocyclealkyl, hydroxyalkyl and (NZ₁Z₂)alkyl;
R₂, R₃, R₄ and R₅ are hydrogen; and
R₆ is phenylmethyl.

8. A compound according to claim 7 selected from the group consisting of
2-[(1-benzyl-1H-indazol-3-yl)oxy]benzoic acid;
2-[(1-benzyl-1H-indazol-3-yl)oxy]-N-[2-(dimethylamino)ethyl]benzamide;
N-[3-(4-{2-[(1-benzyl-1H-indazol-3-yl)oxy]benzoyl}-1-piperazinyl)propyl]-N,N-dimethylamine;
2-[(1-benzyl-1H-indazol-3-yl)oxy]-N-[3-(dimethylamino)propyl]benzamide;
2-[(1-benzyl-1H-indazol-3-yl)oxy]-N-(4-hydroxycyclohexyl)benzamide;
2-[(1-benzyl-1H-indazol-3-yl)oxy]-N-[(1R,2R)-1-hydroxy-2,3-dihydro-1H-inden-2-yl]benzamide;
2-[(1-benzyl-1H-indazol-3-yl)oxy]-N-[1-(hydroxymethyl)butyl]benzamide;
2-[(1-benzyl-1H-indazol-3-yl)oxy]-N-(2-hydroxy-2-phenylethyl)benzamide;
2-[(1-benzyl-1H-indazol-3-yl)oxy]-N-[2-(1-methyl-2-pyrrolidinyl)ethyl]benzamide;
2-[(1-benzyl-1H-indazol-3-yl)oxy]-N-[3-(1H-imidazol-1-yl)propyl]benzamide;
2-[(1-benzyl-1H-indazol-3-yl)oxy]-N′-(4-morpholinyl)benzohydrazide; and
2-[(1-benzyl-1H-indazol-3-yl)oxy]-N-[(2-hydroxycyclohexyl)methyl]benzamide.

9. A compound according to claim 1 wherein
T, X, W, and Y are C;
Z is O;
R₁ is arylalkyl; and
R₆ is arylalkenyl.

10. A compound according to claim 1 wherein
T, X, W, and Y are C;
Z is O;
R₁ is phenylmethyl;
R₂, R₃, R₄ and R₅ are hydrogen; and
R₆ is arylalkenyl wherein the aryl is phenyl.

11. A compound according to claim 10 that is 3-(benzyloxy)-1-[3-phenyl-2-propenyl]-1H-indazole.

12. A compound according to claim 1 wherein
T, X, W, and Y are C;
Z is O;
R₁ is arylalkyl; and
R₆ is heterocyclealkyl.

13. A compound according to claim 1 wherein
T, X, W, and Y are C;
Z is O;
R₁ is phenylmethyl;
R₂, R₃, R₄ and R₅ are hydrogen; and
R₆ is furylmethyl wherein the furyl is substituted with 0, 1 or 2 substituents selected from the group consisting of carboxy, heterocyclecarbonyl, hydroxyalkyl and (NR_{A}R_{B})carbonyl wherein R_{A} and R_{B} are independently selected from the group consisting of hydrogen, cycloalkyl and (NZ₁Z₂)alkyl.

14. A compound according to claim 13 selected from the group consisting of
5-{[3-(benzyloxy)-1H-indazol-1-yl]methyl}-2-furoic acid;
(5-{[3-(benzyloxy)-1H-indazol-1-yl]methyl}-2-furyl)methanol;
3-(benzyloxy)-1-({5-[(4-methyl-1-piperazinyl)carbonyl]-2-furyl}methyl)-1H-indazole;
[1-(5-{[3-(benzyloxy)-1H-indazol-1-yl]methyl}-2-furoyl)-2-pyrrolidinyl]methanol;
5-{[3-(benzyloxy)-1H-indazol-1-yl]methyl}-N-[3-(dimethylamino)propyl]-2-furamide; and
5-{[3-(benzyloxy)-1H-indazol-1-yl]methyl}-N-(4-hydroxycyclohexyl)-2-furamide.

15. A compound according to claim 1 wherein
T, X, and W are C;
Y is N;
Z is O;
R₁ is selected from the group consisting of aryl, arylalkyl and heterocycle;
R₅ is absent; and
R₆ is selected from the group consisting of arylalkenyl, arylalkyl, heterocycle, and heterocyclealkyl.

16. A compound according to claim 1 wherein
T, X, and W are C;
Y is N;
Z is O;
R₁ is heterocycle;
R₅ is absent; and
R₆ is arylalkyl.

17. A compound according to claim 1 wherein
T, X, and W are C;
Y is N;
Z is O;
R₁ is heterocycle selected from the group consisting of furyl, pyridinyl and pyrimidinyl wherein the heterocycle is substituted with 0, 1 or 2 substituents selected from the group consisting of carboxy, haloalkyl, hydroxyalkyl and (NR_{A}R_{B})carbonyl wherein R_{A} and R_{B} are independently selected from the group consisting of hydrogen, arylhydroxyalkyl, heterocyclealkyl, hydroxyalkyl and (NZ₁Z₂)alkyl;
R₂, R₃, and R₄ are hydrogen;
R₅ is absent; and
R₆ is 2-fluorophenylmethyl.

18. The compound according to claim 1 wherein
T, X, and W are C;
Y is N;
Z is O;
R₁ is pyridinyl substituted with nitro;
R₂, R₃, and R₄ are hydrogen;
R₅ is absent; and
R₆ is 2-fluorophenylmethyl.

19. The compound according to claim 18 selected from the group consisting of
1-(2-fluorobenzyl)-3-[(5-nitro-2-pyridinyl)oxy]-1H-pyrazolo[3,4-b]pyridine; and
1-(2-fluorobenzyl)-3-[(3-nitro-2-pyridinyl)oxy]-1H-pyrazolo[3,4-b]pyridine.

20. A method of treating a disorder ameliorated by increasing cGMP levels in a mammal comprising administering to the mammal a therapeutically effective amount of a compound of formula (I).

21. The method according to claim 20 wherein the disorder is selected from the group consisting of cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory and learning disorders.

22. The method according to claim 20 wherein the disorder is sexual dysfunction.

23. The method according to claim 22 wherein the sexual dysfunction is male erectile dysfunction.

24. A method of treating a disorder ameliorated by increasing cGMP levels in a mammal comprising administering to the mammal a therapeutically effective amount of a compound of formula (I) in combination with a pharmaceutically acceptable carrier.

25. The method according to claim 24 wherein the disorder is selected from the group consisting of cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory and learning disorders.

26. The method according to claim 24 wherein the disorder is sexual dysfunction.

27. The method according to claim 26 wherein the sexual dysfunction is male erectile dysfunction.

28. A method of treating a disorder ameliorated by increasing cGMP levels in a mammal comprising administering to the mammal a therapeutically effective amount of a compound of formula (I) in combination with a phosphodiesterase 5 inhibitor.

29. The method according to claim 28 wherein the disorder is selected from the group consisting of cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory and learning disorders.

30. The method according to claim 28 wherein the disorder is sexual dysfunction.

31. The method according to claim 30 wherein the sexual dysfunction is male erectile dysfunction.

32. A method of treating a disorder ameliorated by increasing cGMP levels in a mammal comprising administering to the mammal a therapeutically effective amount of a compound of formula (I) in combination with an adrenergic receptor antagonist.

33. The method according to claim 32 wherein the disorder is selected from the group consisting of cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory and learning disorders.

34. The method according to claim 32 wherein the disorder is sexual dysfunction.

35. The method according to claim 34 wherein the sexual dysfunction is male erectile dysfunction.

36. A method of treating a disorder ameliorated by increasing cGMP levels in a mammal comprising administering to the mammal a therapeutically effective amount of a compound of formula (I) in combination with a dopamine receptor agonist.

37. The method according to claim 36 wherein the disorder is selected from the group consisting of cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory and learning disorders.

38. The method according to claim 36 wherein the disorder is sexual dysfunction.

39. The method according to claim 38 wherein the sexual dysfunction is male erectile dysfunction.
